(19) United States
(12) Patent Application Publication
(10) Pub. No.: US 2026/0242848 A1
(43) Pub. Date: Aug. 20, 2026

(54) METHODS FOR MULTIPLEXED IMAGING AND DETECTION OF TARGETS

(71) Applicant: PRESIDENT AND FELLOWS OF HARVARD COLLEGE, Cambridge, MA (US)

(72) Inventors: Michel I. NOFAL, Cambridge, MA (US); Peng YIN, Cambridge, MA (US); Ryan Berman MCMILLAN, Cambridge, MA (US); Gokul GOWRI, Cambridge, MA (US); Matthew SERRATA, Cambridge, MA (US)

(73) Assignee: PRESIDENT AND FELLOWS OF HARVARD COLLEGE, Cambridge, MA (US)

(21) Appl. No.: 19/157,026

(22) PCT Filed: Feb. 16, 2024
(86) PCT No.: PCT/US2024/016176

§ 371 Date: Aug. 15, 2025

Related U.S. Application Data
(60) Provisional application No. 63/446,581, filed on Feb. 17, 2023.

(51) Int. Cl. C12Q 1/6804; C12Q 1/682

(52) U.S. Cl. CPC C12Q 1/6804; C12Q 1/682

(57) ABSTRACT

The disclosure relates generally to methods, compositions, and kits for imaging (e.g., multiplexed imaging with DNA-based signal amplification) of target molecules, e.g., biomolecules in a sample such as in cells and tissues.

### CROSS-REFERENCE TO RELATED APPLICATIONS

This application claims benefit under 35 U.S.C. § 119(e) of U.S. Provisional Application No. 63/446,581, filed Feb. 17, 2023, contents of which are incorporated herein by reference in their entirety.

### GOVERNMENT SUPPORT

This invention was made with government support under GM133052 and MH124606 and GM124401 and CA255133 awarded by National Institutes of Health (NIH). The government has certain rights in this invention.

### TECHNICAL FIELD

The disclosure relates generally to compositions, kits and methods for imaging (e.g., multiplexed imaging with DNA-based signal amplification) of target molecules, e.g., biomolecules in a sample such as in cells and tissues.

### BACKGROUND

There is a need for distinguishably and simultaneously visualizing (i.e., in a multiplex format) targets in a sample. The present disclosure addresses these needs.

### SUMMARY

The methods, compositions and kits described herein have applicability in, for example, high-throughput screening assays such as drug screening assays. The methods, compositions and kits described herein allow analysis of large populations of cells (~1,000-10,000) or tissue samples in an ultra-multiplexed format while imaging using standard confocal or epi-fluorescence microscope. Screening large numbers of targets such as proteins from the same sample in a high-throughput manner can provide information about new drugs or modifiers while providing cellular heterogeneity information. The large-scale screening of tissue samples with high-throughput and ultra-multiplexed imaging capabilities can be useful in pathology analysis, for example, in a hospital or other service provider setting.

It is noted that the methods, compositions and kits described herein are not limited to high-throughput screening assays and have broad applicability in, for example, for multiplexed characterization of biological samples. For example, multiplexed ELISA can be used to characterize human samples in the clinical setting; multiplexed Western Blots can be used to characterize signaling pathways in cancer cell lines or in mouse models of cancer; multiplexed immunofluorescence can be used to study cellular heterogeneity or to map tissues.

The methods, compositions and kits described herein can be used to identify the location of a target, e.g., a plurality of targets within a sample or relative to other targets in the sample. Additionally, the compositions, kits and methods described herein can be used for multiplex detection or imaging of targets in a sample.

In one aspect provided herein is a method for imaging a target in a sample. In embodiments of the various aspects described herein, the sample is imaged using confocal or epi-fluorescence microscopy.

In some embodiments, the method comprises:
(a) contacting a sample being tested for the presence of one or more targets with one or more target-specific target-binding molecules, wherein each target-binding molecule is linked to a barcode strand, and wherein target-binding molecules of different specificity are linked to different barcode strands;
(b) optionally removing unbound target-binding molecules;
(c) contacting the sample with amplifier strands comprising a barcode binding domain and a plurality of adapter binding domains, wherein the barcode binding domain comprises a nucleotide sequence that is substantially complementary to a barcode strand;
(d) optionally removing unbound amplifier strands;
(e) contacting the sample with a first set of adaptor strands bound/hybridized with imager strands, wherein the adaptor strands comprise an amplifier binding domain and at least one imager binding domain, wherein the amplifier domain comprises a nucleotide complementary to a nucleotide sequence of an adapter binding domain, and wherein the imager strands comprising a detectable label and a nucleotide sequence that is complementary to a nucleotide sequence of an imager binding domain;
(f) optionally removing unbound adapter strands and imager strands;
(g) imaging the sample to detect location of bound imager strands; and
(h) extinguishing a detectable signal from the detectable labels of the bound imager strands;
(i) optionally, repeating steps (e)-(h) at least once with a second set of adaptor strands bound/hybridized with imager strands and having a unique nucleotide sequence relative to the nucleotide sequences of the adaptor strands in the first set.

In some embodiments, the method comprises:
(a) contacting a sample being tested for the presence of one or more targets with one or more target-specific target-binding molecules, wherein each target-binding molecule is linked to a barcode strand, and wherein target-binding molecules of different specificity are linked to different barcode strands;
(b) optionally removing unbound target-binding molecules;
(c) contacting the sample with amplifier strands comprising a barcode binding domain and a plurality of adapter binding domains, wherein the barcode binding domain comprises a nucleotide sequence that is substantially complementary to a barcode strand;
(d) optionally removing unbound amplifier strands;
(e) contacting the sample with a first set of adaptor strands comprising an amplifier binding domain and at least one imager binding domain, wherein the amplifier domain comprises a nucleotide complementary to a nucleotide sequence of an adapter binding domain;
(f) optionally removing unbound adapter strands;
(g) contacting the sample with imager strands comprising a detectable label and a nucleotide sequence that is complementary to a nucleotide sequence of an imager binding domain;
(h) optionally removing unbound imager strands; and
(i) imaging the sample to detect location of bound imager strands;
(j) extinguishing a detectable signal from the detectable labels of the bound imager strands; and
(k) repeating steps (e)-(j) at least once with a second set of adaptor strands bound/hybridized with imager strands and having a unique nucleotide sequence relative to the nucleotide sequences of the adaptor strands in the first set.

In some embodiments of any one of the aspects described herein, the step of extinguishing the signal from the bound imager strands comprises removing the adaptor strands from the amplifier strands they are hybridized to, or removing the detectable label from the bound imager strands, or modifying the detectable label. For example, the step of extinguishing the signal from the bound imager strands comprises removing the bound adaptor strands from the amplifier strands they are hybridized to by altering temperature, by altering buffer conditions and/or addition of a complementary nucleic acid strand. In some embodiments of any one of the aspects described herein, the strands are removed from the amplifier strand by addition of a denaturant, increasing temperature, addition of a complementary nucleic acid strand, and/or decreasing salt concentration. For example, the adaptor strands are removed from the amplifier strand by addition of a denaturant selected from the group consisting of formamide, urea, and DMSO. In some embodiments of any one of the aspects described herein, the adaptor strands are removed from the amplifier strand by addition of complementary nucleic acid strands, wherein the nucleic acid strands comprise a nucleotide sequence substantially complementary to amplifier binding domain of the adaptor strand or the adaptor binding domain of the amplifier strand. In some embodiments of any one of the aspects described herein, the step of extinguishing the signal from the bound imager strands comprises removing or modifying the detectable label without removing the adaptor strands. In some embodiments of any one of the aspects described herein, the step of extinguishing the signal from the bound imager strands comprises photobleaching. In some embodiments of any one of the aspects described herein, the step of extinguishing the signal from the bound imager strands comprises cleaving the detectable label from the imager strand. For example, cleaving the detectable label from the imager strand comprises enzymatic cleavage, chemical cleavage or photo cleavage.

The methods described herein can be used in a multiplex format to detect two or more targets. Accordingly, in some embodiments of any one of the aspects described herein, at least one adapter strand in the first set of the adaptor strands binds/hybridizes with a first amplifier strand and at least one other adapter strand in the first set of the adaptor strands binds/hybridizes with a second amplifier strand, and wherein the first and second amplifier strand are bound/hybridized to barcode strands linked to target-binding molecules of different specificity (i.e., target-binding molecules bind to different targets).

In some embodiments of any one of the aspects described herein, at least one adapter strand in the second set of the adaptor strands binds/hybridizes with a first amplifier strand and at least one other adapter strand in the second set of the adaptor strands binds/hybridizes with second amplifier strand, and wherein the first and second amplifier strand are bound/hybridized to barcode strands linked to target-binding molecules of different specificity (i.e., target-binding molecules bind to different targets).

In some embodiments, at least one imager strand binds to an adaptor strand binds/hybridizes with a first amplifier strand and at least one other adaptor strand binds/hybridizes with a second amplifier strand, and wherein the first and second amplifier strand are bound/hybridized to barcode strands linked to target-binding molecules of different specificity (i.e., target-binding molecules bind to different targets), and wherein the detectable labels of the imager strands are different.

In some embodiments, a melting temperature (Tm) of imager strand binding/hybridizing with the adaptor strand is lower than a melting temperature of the amplifier strand binding/hybridizing with the barcode strand and the imager strand binding/hybridizing with the adaptor strand. For example, melting temperature of imager strand binding/hybridizing with the adaptor strand is at least about 5° C. lower than a melting temperature of the amplifier strand binding/hybridizing with the barcode strand and the imager strand binding/hybridizing with the adaptor strand.

Without limitations, the amplifier binding domain and the imager domains of an amplifier strands can be arranged in any desired orientation position. Accordingly, in some embodiments, the amplifier binding domain of the adaptor strand is at 5′ of the imager binding domain. In some other embodiments, the amplifier binding domain of the adaptor strand is at 3′ of the imager binding domain.

Similarly, the barcode binding domain and the adaptor binding domains of the amplifier strands can be arranged in any desired orientation. For example, the barcode binding domain of the amplifier strand can be 5′ of the plurality of adapter binding domains. Alternatively, the barcode binding domain of the amplifier strand is 3′ of the plurality of adapter binding domains.

In some embodiments, the method comprises:
(a) contacting a sample being tested for the presence of one or more targets with one or more target-specific target-binding molecules, wherein each target-binding molecule is linked to a barcode strand, and wherein target-binding molecules of different specificity are linked to different barcode strands,
(b) optionally removing unbound target-binding molecules,
(c) contacting the sample with amplifier strands comprising a barcode binding domain, wherein the barcode binding domain comprises a nucleotide sequence that is substantially complementary to a barcode strand,
(d) optionally removing unbound amplifier strands,
(e) contacting the sample with a first set of imager strands comprising a detectable label and a nucleotide sequence that is substantially complementary to a nucleotide sequence of an imager binding domain,
(f) optionally removing unbound imager strands,
(g) imaging the sample to detect location of bound labeled imager strands,
(h) extinguishing a detectable signal from the bound labeled imager strands, and
(i) optionally, repeating steps (e)-(h), each time with second set of imager strands having a unique nucleotide sequence relative to the imager strands of the first set.

In some embodiments, the step of extinguishing the signal from the bound imager strands comprises removing the imager strands from the amplifier strands they are hybridized to, or removing the detectable label from the bound imager strands, or modifying the detectable label. For example, the step of extinguishing the signal from the bound imager strands comprises removing the bound imager strands from the amplifier strands they are hybridized to by altering temperature, by altering buffer conditions and/or addition of a complementary nucleic acid strand. For example, the imager strands can be removed from the amplifier strand by addition of a denaturant, increasing temperature, addition of a complementary nucleic acid strand, and/or decreasing salt concentration. In some embodiments, the imager strands can be removed from the amplifier strand by addition of a denaturant selected from the group consisting of formamide, urea, and DMSO. In some embodiments, the imager strands are removed from the amplifier strand by addition of complementary nucleic acid strands, wherein the nucleic acid strands comprise a nucleotide sequence substantially complementary to amplifier binding domain of the imager strand or the imager binding domain of the amplifier strand. In some embodiments, the step of extinguishing the signal from the bound imager strands comprises removing or modifying the detectable label without removing the imager strands. For example, the step of extinguishing the signal from the bound imager strands comprises photobleaching. In some embodiments, the step of extinguishing the signal from the bound imager strands comprises cleaving the detectable label from the imager strand. For example, cleaving the detectable label from the imager strand comprises enzymatic cleavage or photo cleavage.

As described herein, the methods can be used in a multiplex format to detect two or more targets. Accordingly, in some embodiments of any one of the aspects described herein, at least one imager strand in the first set of the imager strands binds/hybridizes with a first amplifier strand and at least one other imager strand in the first set of the imager strands binds/hybridizes with second amplifier strand, and wherein the first and second amplifier strand are bound/hybridized to barcode strands linked to target-binding molecules of different specificity (i.e., target-binding molecules bind to different targets), and wherein the detectable labels are different.

In some embodiments of any one of the aspects described herein, at least one imager strand in the second set of the imager strands binds/hybridizes with a first amplifier strand and at least one other imager strand in the second set of the adaptor strands binds/hybridizes with a second amplifier strand, and wherein the first and second amplifier strand are bound/hybridized to barcode strands linked to target-binding molecules of different specificity (i.e., target-binding molecules bind to different targets) and wherein the detectable labels are different.

In some embodiments of any one of the aspects described herein, a melting temperature of imager strand binding/hybridizing with the amplifier strand is lower than the amplifier strand binding/hybridizing with the barcode strand. For example, a melting temperature of imager strand binding/hybridizing with the amplifier strand is at least about 5° C. lower than the amplifier strand binding/hybridizing with the barcode strand.

It is noted that the barcode binding domain and the imager binding domains of an amplifier strands can be arranged in any desired orientation. Accordingly, in some embodiments, the barcode binding domain of the amplifier strand is 5′ of the plurality of imager binding domains. In some other embodiments, the barcode binding domain of the amplifier strand is 3′ of the plurality of imager binding domains.

Embodiments of the various aspects described herein include imager strands comprising a detectable label. The detectable label can be attached at the 5′-end, 3′-end, or at an internal position. In some embodiments, the detectable label is attached at the 5′-end of the imager strand it is attached to. In some other embodiments, the detectable label is attached at the 3′-end of the imager strand it is attached to.

In some embodiments of any one of the aspects described herein, the imager strand comprises, at its end the detectable label is attached to, at least two nucleotides that do not hybridized with the strand the imager strand is bound/hybridized to.

In some embodiments of any one of the aspects described herein, the unbound imager strands are partially double-stranded. For example, the imager strands are molecular beacons or comprise a hairpin secondary structure that is self-quenching.

An imager strand described herein can comprise multiple (e.g., 2such as, 3, 4, 5, 6, 7, 8, 9, 10 or more) detectable labels. In some embodiments, the imager strands comprise at least two detectable labels. For example, the imager strands comprise a first detectable label at their 5′-end and a second detectable label at their 3′-end.

Similar to the imager strands, unbound barcode strands can be partially double-stranded. In some embodiments, the unbound barcode strands can comprise a hairpin secondary structure.

Without limitations, a barcode can be linked to target-binding molecule by any position in the barcode strand. For example, the barcode strand can be linked to the target-binding molecule via its 5′-end. Alternatively, the barcode strand can be linked to the target-binding molecule via its 3′-end.

Some exemplary target-binding molecules include, but are not limited to, an antibody, antibody fragment (e.g., antigen binding portion of an antibody), a nucleic acid, a receptor, a ligand for a receptor, an antigen, or an enzyme. In some embodiments, the target-binding molecule is an antibody, antigen binding portion of an antibody, or a nucleic acid.

For multiplex imaging, the sample is contacted with more than one target-binding molecule in step (a).

In yet another aspect, provide herein is a composition comprising: target-specific target binding molecules linked to a barcode strand, amplifier strands, imager strands, and optionally, adaptor strands described herein.

In some embodiments, the composition comprises: (i) a first target-specific target binding molecule linked to a first barcode strand; (ii) a first amplifier strand comprising a barcode binding domain and a plurality of adapter binding domains, wherein the barcode binding domain comprises a nucleotide sequence that is substantially is substantially complementary to the first barcode strand; (iii) at least one first adaptor strand comprising an amplifier binding domain and at least one imager binding domain, wherein the amplifier domain comprises a nucleotide complementary to a nucleotide sequence of the adapter binding domain of the first amplifier strand; and (iv) at least one first imager strand comprising a first detectable label and a nucleotide sequence that is complementary to a nucleotide sequence of the imager binding domain of the first adaptor strand. In some further embodiments of this, the composition further comprises: (i) a second target-specific target binding molecule linked to a second barcode strand, wherein the second target-binding molecule binds to a target that is different from the first target-binding molecule; (ii) a second amplifier strand comprising a barcode binding domain and a plurality of adapter binding domains, wherein the barcode binding domain comprises a nucleotide sequence that is substantially is substantially complementary to the second barcode strand; (iii) at least one second adaptor strand comprising an amplifier binding domain and at least one imager binding domain, wherein the amplifier domain comprises a nucleotide complementary to a nucleotide sequence of the adapter binding domain of the second amplifier strand; and (iv) at least one second imager strand comprising a second detectable label and a nucleotide sequence that is complementary to a nucleotide sequence of the imager binding domain of the second adaptor strand, and wherein the first and second detectable labels are different.

In some embodiments, the composition comprises: (i) a first target-binding molecule linked to a first barcode strand; (ii) a first amplifier strands comprising a barcode binding domain, wherein the barcode binding domain comprises a nucleotide sequence that is substantially complementary to the first barcode strand; and (iii) a first imager strand comprising a first detectable label and a nucleotide sequence that is substantially complementary to a nucleotide sequence of the first imager binding domain. In some further embodiments of this, the composition further comprises: (i) a second target-binding molecule linked to a second barcode strand, wherein the second target-binding molecule binds to a target that is different from the first target-binding molecule; (ii) a second amplifier strands comprising a barcode binding domain, wherein the barcode binding domain comprises a nucleotide sequence that is substantially complementary to the second barcode strand; and (iii) a second imager strand comprising a second detectable label and a nucleotide sequence that is substantially complementary to a nucleotide sequence of the second imager binding domain, and wherein the first and second detectable labels are different.

In another aspect, provided herein is a kit comprising a composition described herein. For example, the kit comprises: target-specific target binding molecules linked to a barcode strand, amplifier strands, imager strands, and optionally, adaptor strands described herein.

In some embodiments, the kit comprises: (i) a first target-specific target binding molecule linked to a first barcode strand; (ii) a first amplifier strand comprising a barcode binding domain and a plurality of adapter binding domains, wherein the barcode binding domain comprises a nucleotide sequence that is substantially is substantially complementary to the first barcode strand; (iii) at least one first adaptor strand comprising an amplifier binding domain and at least one imager binding domain, wherein the amplifier domain comprises a nucleotide complementary to a nucleotide sequence of the adapter binding domain of the first amplifier strand; and (iv) at least one first imager strand comprising a first detectable label and a nucleotide sequence that is complementary to a nucleotide sequence of the imager binding domain of the first adaptor strand. In some further embodiments of this, the kit further comprises (i) a second target-specific target binding molecule linked to a second barcode strand, wherein the second target-binding molecule binds to a target that is different from the first target-binding molecule; (ii) a second amplifier strand comprising a barcode binding domain and a plurality of adapter binding domains, wherein the barcode binding domain comprises a nucleotide sequence that is substantially is substantially complementary to the second barcode strand; (iii) at least one second adaptor strand comprising an amplifier binding domain and at least one imager binding domain, wherein the amplifier domain comprises a nucleotide complementary to a nucleotide sequence of the adapter binding domain of the second amplifier strand; and (iv) at least one second imager strand comprising a second detectable label and a nucleotide sequence that is complementary to a nucleotide sequence of the imager binding domain of the second adaptor strand, and wherein the first and second detectable labels are different.

In some embodiments, the kit comprises: (i) a first target-binding molecule linked to a first barcode strand; (ii) a first amplifier strands comprising a barcode binding domain, wherein the barcode binding domain comprises a nucleotide sequence that is substantially complementary to the first barcode strand; and (iii) a first imager strand comprising a first detectable label and a nucleotide sequence that is substantially complementary to a nucleotide sequence of the first imager binding domain. In some further embodiments of this, the kit further comprises: (i) a second target-binding molecule linked to a second barcode strand, wherein the second target-binding molecule binds to a target that is different from the first target-binding molecule; (ii) a second amplifier strands comprising a barcode binding domain, wherein the barcode binding domain comprises a nucleotide sequence that is substantially complementary to the second barcode strand; and (iii) a second imager strand comprising a second detectable label and a nucleotide sequence that is substantially complementary to a nucleotide sequence of the second imager binding domain, and wherein the first and second detectable labels are different.

In some embodiments of the various aspects described herein, a melting temperature of the imager strand binding/hybridizing with the adaptor strand is lower than a melting temperature of the amplifier strand binding/hybridizing with the barcode strand and the imager strand binding/hybridizing with the adaptor strand. For example, a melting temperature of imager strand binding/hybridizing with the adaptor strand is at least about 5° C. lower than a melting temperature of the amplifier strand binding/hybridizing with the barcode strand and the imager strand binding/hybridizing with the adaptor strand.

In some embodiments of the various aspects described herein, the amplifier binding domain of the adaptor strand is at 5′ of the imager binding domain. In some other embodiments of the kit, the amplifier binding domain of the adaptor strand is at 3′ of the imager binding domain.

In some embodiments of the various aspects described herein, the barcode binding domain of the amplifier strand is 5′ of the plurality of adapter binding domains. In some other embodiments of the kit, the barcode binding domain of the amplifier strand is 3′ of the plurality of adapter binding domains.

In some embodiments of the various aspects described herein, a melting temperature of the imager strand binding/hybridizing with the adaptor strand is from about 35° C. to about 45° C.

In some embodiments of the various aspects described herein, a melting temperature of the adaptor strand binding/hybridizing with the amplifier strand is at least 50° C. or higher.

In some embodiments of the various aspects described herein, the amplifier strand comprises at least 5 (e.g., 5, 6, 7, 8, 9, 10, 11, 12, 13, 14, 15 or more) adaptor binding domains. For example, the amplifier strand comprises 10 adaptor binding domains.

In some embodiments of the various aspects described herein, the imager strand is hybridized with the adaptor strand. In some embodiments of the various aspects described herein, the adaptor strand is hybridized with the amplifier strand. In some embodiments of the various aspects described herein, the imager strand is hybridized with the adaptor strand and the adaptor strand is hybridized with the amplifier strand.

In some embodiments of the various aspects described herein, a melting temperature of imager strand binding/hybridizing with the amplifier strand is lower than the amplifier strand binding/hybridizing with the barcode strand. In some other embodiments of the kit, a melting temperature of imager strand binding/hybridizing with the amplifier strand is at least about 5° C. lower than the amplifier strand binding/hybridizing with the barcode strand.

In some embodiments of the various aspects described herein, the barcode binding domain of the amplifier strand is 5′ of the plurality of imager binding domains. In some other embodiments, the barcode binding domain of the amplifier strand is 3′ of the plurality of imager binding domains.

In some embodiments of the various aspects described herein, a melting temperature of the imager strand binding/hybridizing with the amplifier strand is from about 35° C. to about 45° C.

In some embodiments of the various aspects described herein, the amplifier strand comprises at least 5 (e.g., 5, 6, 7, 8, 9, 10, 11, 12, 13, 14, 15 or more) imager binding domains.

In some embodiments of the various aspects described herein, the imager strand is hybridized with the amplifier strand.

In some embodiments of the various aspects described herein, a melting temperature of the amplifier strand binding/hybridizing with the barcode strand is at least 50° C. or higher.

In some embodiments of the various aspects described herein, the detectable label is attached at the 5′-end of the imager strand it is attached to.

In some embodiments of the various aspects described herein, detectable label is attached at the 3′-end of the imager strand it is attached to.

In some embodiments of the various aspects described herein, the imager strand comprises, at its end the detectable label is attached to, at least two nucleotides that do not hybridized with the strand the imager strand is bound/hybridized to.

In some embodiments of the various aspects described herein, the unbound imager strands are partially double-stranded.

In some embodiments of the various aspects described herein, imager strands are molecular beacons or comprise a hairpin secondary structure.

In some embodiments of the various aspects described herein, the imager strands are molecular beacons or comprise a hairpin secondary structure that is self-quenching.

In some embodiments of the various aspects described herein, wherein the imager strands comprise multiple (e.g., two such as, 3, 4, 5, 6, 7, 8, 9 10 or more) detectable labels.

In some embodiments of the various aspects described herein, imager strands comprise a first detectable label at their 5′-end and a second detectable label at their 3′-end.

In some embodiments of the various aspects described herein, the imager strand is from about 10 nucleotides to about 15 nucleotides in length, e.g., about 15 nucleotides in length.

In some embodiments of the various aspects described herein, unbound barcode strands are partially double-stranded.

In some embodiments of the various aspects described herein, unbound barcode strands comprise a hairpin secondary structure.

In some embodiments of the various aspects described herein, the barcode strand is linked to the target-binding molecule via its 5′-end. In some other embodiments, the barcode strand is linked to the target-binding molecule via its 3′-end.

In some embodiments of the various aspects described herein, the target-binding molecule is an antibody, a nucleic acid, a receptor, a ligand for a receptor, an antigen, or an enzyme.

In some embodiments of the various aspects described herein, the target-binding molecule is an antibody or antibody fragment (e.g., antigen binding portion of an antibody) or a nucleic acid.

In some embodiments of the various aspects described herein, the target-binding molecule is an antibody or antibody fragment (e.g., antigen binding portion of an antibody).

To reduce or inhibit off-target binding of imager strands, i.e., binding to non-complementary sequences, the imager strands can be added to the sample with blocker strands. Also referred to as cross-talk blockers herein, the blocker strands are similar, e.g., identical in sequence to the imager strands but don't comprise a detectable label. Thus, in some embodiments, the step of contacting the sample with the imager strands is in presence of blocker strands. Without wishing to be bound by a theory, relative concentration of the blocker strands to the imager strands can be adjusted to reduce or inhibit off-target binding of imager strand. For example, the blocker can be added at a low concentration to block unused amplifier strands from off-target binding. Accordingly, in some embodiments, the step of contacting the sample with the imager strands is in presence of blocker strands, where a concentration of the blocker strands is lower than a concentration of the imager strands. For example, concentration of the blocker strands is at least about 95% or lower (e.g., about 90%, about 85%, about 80%, about 75%, about 70%, about 65%, about 60%, about 55%, about 50%, about 45%, about 40%, about 35%, about 30%, about 25%, about 20%, about 15%, about 10% or lower) than the concentration of the imager strands. It is noted that the blocker strands can be added to the sample, prior to, simultaneously with or after contacting with the imager strands.

These and other embodiments will be described in greater detail herein.

### BRIEF DESCRIPTION OF THE DRAWINGS

This patent or application file contains at least one drawing executed in color. Copies of this patent or patent application publication with color drawings will be provided by the Office upon request and payment of the necessary fee.

FIG. 1 is a schematic representation of a protocol or method for multiplexed immunofluorescence according to an embodiment of the method described herein. First, DNA barcoded antibodies are added to the sample. Then, DNA amplifiers are added. Finally, DNA imagers are added in groups, and the sample is imaged. After imaging, old DNA imagers are washed out and new DNA imagers are applied. Because the interaction between the DNA barcodes and DNA amplifiers are stable, this can be repeated for a very large number of targets.

FIG. 2 shows results of an exemplary imaging study using an exemplary embodiment of the method described herein Formalin-fixed human kidney tissue was stained with DNA-conjugated rabbit anti-E-cadherin antibody and imaged with anti-rabbit secondary antibody DNA-conjugated mouse anti-rabbit antibody (magenta), or with DNA imagers. DNA imagers bound directly to the DNA barcode without a DNA amplifier (top) or to a DNA amplifier with ten imager binding sites (bottom).

FIG. 3 shows results of an exemplary imaging study using another exemplary embodiment of the method described herein. HeLa cells were fixed with 4% paraformaldehyde for 15 minutes, stained with rabbit anti-tubulin antibody, DNA-conjugated mouse anti-rabbit antibody, and imaged with a donkey anti-mouse tertiary antibody (magenta) or DNA-amplifier and DNA-imagers. Pre-complexed universal imagers (bottom) perform as well as standard imagers (top). Scale bars are 10 μm.

FIG. 4 shows results of an exemplary imaging study using yet another exemplary of embodiment of the method described herein. Hela cells were fixed with 4% paraformaldehyde for 15 minutes, stained with rabbit anti-tubulin antibody, DNA-conjugated mouse anti-rabbit antibody, and imaged with a donkey anti-mouse tertiary antibody (magenta) or DNA-amplifier and DNA-imagers. Universal imagers conjugated with one dye molecule (top) were compared with universal imagers conjugated with two (bottom). Signal is increased roughly 50%. Scale bars are 10 μm.

FIGS. 5A and 5B are schematic representations of DNA-based reagents assembled on DNA-conjugated antibodies with single-fluorophore imager strands (FIG. 5A) and double-fluorophore imager strands (FIG. 5B) according to exemplary embodiments of the disclosure. The 25-nt interaction between the DNA barcode and the DNA amplifier has a melting temperature of roughly 55° C. The 17-nt interaction between the imager adaptor strands and the DNA amplifier have a lower melting temperature, 43° C., enabling imager stripping without disruption of the barcode-amplifier interaction. The two components of the DNA imager, the adaptor and the universal imager, are held in complex by a 25-nt interaction such that when imagers are stripped, these two strands remain together, preventing aberrant binding of the universal imager to the sample.

FIG. 6 show results of an imaging study according to an embodiment of the disclosure demonstrating comparable signal amplification as using a secondary antibody. Scale bars are 10 μm.

FIG. 7 shows results of a multiplex imaging study of 6 protein targets according to an embodiment of the disclosure. Scale bars are 10 μm.

FIG. 8 shows results of a multiplex imaging study of 24 protein targets according to an embodiment of the disclosure. Scale bars are 10 μm.

FIG. 9 show results of an imaging study according to an embodiment of the disclosure demonstrating cross-talk between imaging channels can be reduced or inhibited with addition of blocker strands.

FIGS. 10A and 10B show the multiplexing method described herein enable completer signal removal with minimal decay over 20 rounds. FIG. 10A, scale bars are 10 μm. FIG. 10B, normalized signal intensity over 20 rounds.

FIGS. 11A and 11B show the multiplexing method described herein enable multiple rounds of RNA imaging.

### DETAILED DESCRIPTION

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

In one aspect, the disclosure relates to methods for multiplexed imaging with DNA-based signal amplification of biomolecules in cells and tissues. In some embodiments, proteins are imaged using antibodies conjugated with DNA barcodes. DNA amplifier strands bind specifically to these barcodes; these DNA amplifier strands contain an array of binding sites for DNA imagers. DNA imagers are composed of multiple oligonucleotides: the DNA-imager adaptor, which binds to the DNA amplifier, and the Universal Imager, which is conjugated to a fluorescent dye molecule; DNA imagers bind to DNA amplifiers. As an ensemble, these DNAs decorate each biomolecular target to be imaged with many fluorophores, thereby achieving signal amplification.

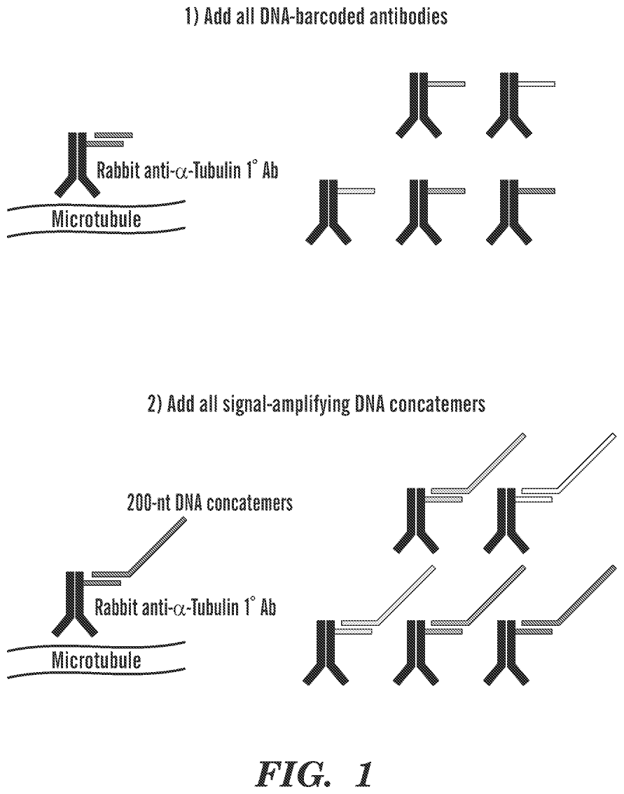

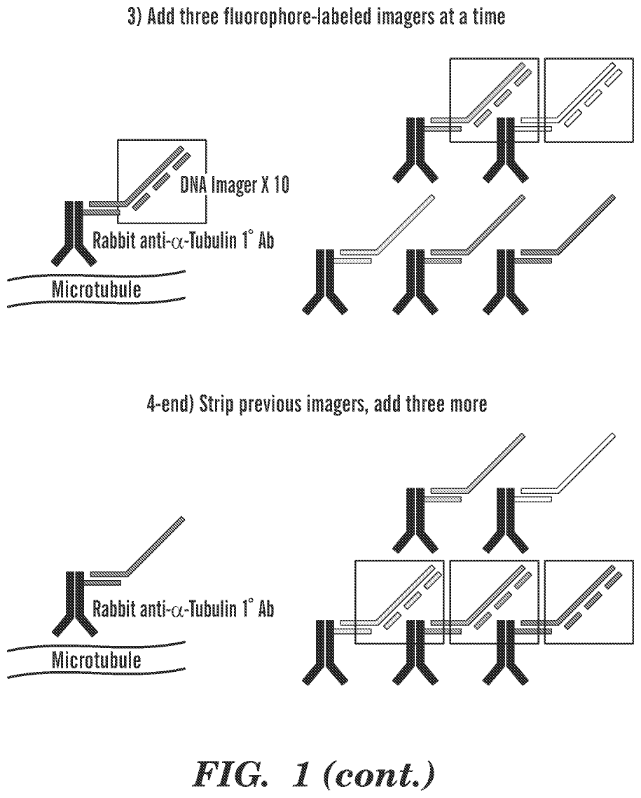

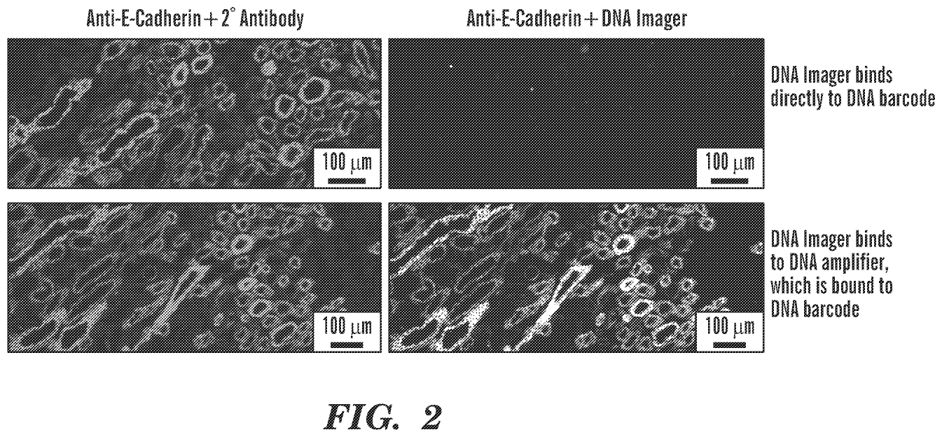

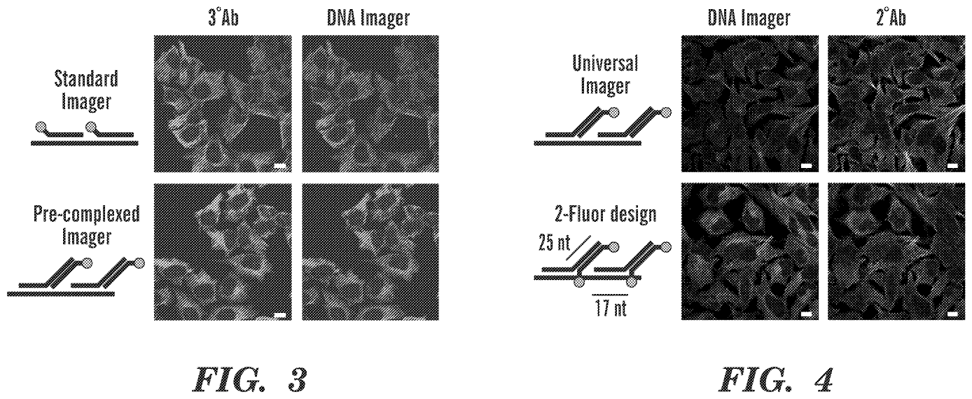

An exemplary method is shown in FIG. 1. To perform multiplexed imaging, first, all probes targeting biomolecules of interest (for example, DNA-barcoded antibodies targeting proteins) are added to the sample. Once these primary probes have been added, all DNA amplifier strands are added. These DNA amplifier strands are added in conditions that limit aberrant binding (for example, high formamide and high temperature). Finally, a fiducial marker, to be used for image registration between imaging rounds (for example, DAPI or fluorescent beads), is added. At this point, the sample is ready to be imaged and is positioned on the microscope. DNA imagers are then added in groups. In some cases, three or four DNA imagers are added per imaging round. In other cases, if a microscope can image more than four channels simultaneously, more DNA imagers can be added per imaging round. Once DNA imagers are added, the sample is imaged.

After imaging, dye molecules are removed. In some embodiments, DNA imagers are stripped with solutions containing formamide, which destabilizes the interaction between two DNA oligonucleotides. In these cases, it is possible to disrupt the binding of DNA imagers to DNA amplifiers without disrupting the binding of DNA amplifiers to DNA barcodes or the binding of the independent components that comprise the DNA imager to each other. This selective disruption of the DNA amplifier-DNA imager interaction is made possible by engineering different interactions to have different stabilities. For example, in some embodiments, the DNA barcode-DNA amplifier interaction spans 25 base-pairs; the DNA-imager adaptor-Universal Imager interaction also spans 25 base-pairs; and the DNA amplifier-DNA imager interactions only includes 17 base-pairs. Due to the different stabilities of these interacting DNAs, it is possible to wash the sample with a formamide-containing buffer (for example, 50% formamide in 2× saline-sodium citrate buffer) that destabilizes some interactions while others remain stable.

In some embodiments, dye molecules can be linked to DNA imagers using disulfide bonds; these dye molecules can be removed by washing the sample with a reducing agent (for example, tris(2-carboxyethyl)phosphine). In some embodiments, dye molecules can be linked to DNA imagers using photo-cleavable linkers; these dye molecules can be removed with exposure to ultraviolet light.

In some embodiments, this amplification scheme can be applied to achieve multiplexed imaging of RNAs and DNAs. In these cases, probes hybridizing to RNA and DNA targets can include overhangs that serve as DNA barcodes.

DNA amplifiers contain two parts: (i) a sequence which binds to the DNA barcode associated with the primary probe or antibody and (ii) a repeating sequence which serves as a signal amplifier. The set of sequences comprising the DNA barcodes and their reverse complements are designed to be highly stable, specific, and orthogonal. The repeating sequences and their reverse complements are designed to be stable at 22° C. in the absence of formamide, but less stable than the DNA barcodes and their reverse complements, such that they can be removed without disrupting barcode-amplifier interactions; repeating sequences and their reverse complements are also designed to be specific and orthogonal. In some embodiments, a DNA amplifier might have one binding site that enables hybridization to the DNA barcode (25 nucleotides) and ten imager binding sites (17 nucleotides each); this enables ten DNA imagers to bind to each DNA barcode, thereby achieving ten-fold amplification.

In some embodiments, this signal amplification strategy can enable visualization of targets that are too scarce to visualize without amplification. In some embodiments, this signal amplification strategy can increase signal-to-noise ratio, where noise derives from autofluorescent background or aberrant DNA imager binding, for example. In some embodiments, this signal amplification can reduce the time required for image acquisition, enabling increased throughput.

Fluorophore-conjugated DNA oligonucleotides can be used to associate fluorescent dye molecules with target DNA strands in a programmable manner. The number of unique fluorophore-conjugated DNA oligonucleotides required is equal to the number of channels imaged. For example, to acquire a multiplexed image of 30 target biomolecules using 3 fluorescent channels, 30 DNA imagers would be required. If DNA imagers are comprised of only one DNA strand that binds directly to the DNA amplifier strand, then 30 fluorophore-conjugated oligonucleotides are required to image 30 targets. These oligonucleotides are expensive due to the cost of the fluorescent dye molecule and the requirement for HPLC purification to prevent contaminants.

In one aspect, the disclosure employs a strategy that dramatically reduces the number of fluorophore-conjugated oligonucleotides required, thereby reducing costs. Rather than using DNA imagers comprised of only one DNA strand, two or more are used. One of these is a fluorophore-conjugated DNA oligonucleotide, termed a “Universal Imager.” The other is an adaptor oligonucleotide, binding to the universal imager at one end and to the DNA amplifier strand at the other. Universal imager and adaptor are mixed together by the user before adding the complex to the sample, which has already stained with primary probes and amplifiers. Universal imager is added in excess, and after universal imager and adaptor are allowed to hybridize, a quencher DNA strand, the reverse complement of the universal imager, is added to block the single-stranded universal imager from binding aberrantly to the sample.

These adaptor nucleotides can be synthesized cheaply, without the need for labor-intensive purification methods, including those involving high-performance liquid chromatography (HPLC) or polyacrylamide gel electrophoresis (PAGE). Using this strategy, the number of unique fluorophore-conjugated DNA oligonucleotides required is equal to the number of channels available on the microscope being used (usually 3 or 4 for standard fluorescence microscopes). For example, to acquire a multiplexed image of 30 target biomolecules using 3 fluorescent channels, only 3 universal imager oligonucleotides are required, as well as 30 low-cost adaptor oligonucleotides and 3 quencher oligonucleotides.

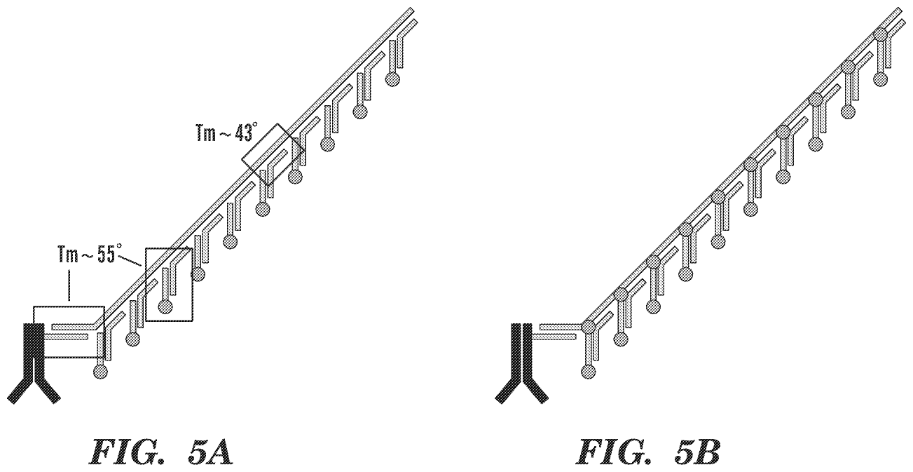

Because only a fixed number of fluorophore-labeled oligonucleotides are required, additional functionalities can be added for a fixed cost (cost does not scale with number of target biomolecules). In some embodiments, two dye molecules can be added to each imager oligonucleotide. In some embodiments, the dye molecule can be added to the oligonucleotide via a linker containing a disulfide bond, such that fluorescence can be removed with a reductant, or via a linker containing a photocleavable linker, such that fluorescence can be removed with ultraviolet light. FIGS. 5A and 5B are schematics illustrating the full assembly of DNA-based reagents on antibodies used to image proteins in multiplexed fashion according to some exemplary embodiments.

The various aspects of the disclosure are described in more detail below.

### Methods

In some embodiments, the method comprises: (a) contacting a sample being tested for the presence of one or more targets with one or more target-specific target-binding molecules, wherein each target-binding molecule is linked to a barcode strand, and wherein target-binding molecules of different specificity are linked to different barcode strands; (b) optionally removing unbound target-binding molecules; (c) contacting the sample with amplifier strands comprising a barcode binding domain and a plurality of adapter binding domains, wherein the barcode binding domain comprises a nucleotide sequence that is substantially complementary to a barcode strand; (d) optionally removing unbound amplifier strands; (e) contacting the sample with a first set of adaptor strands bound/hybridized with imager strands, wherein the adaptor strands comprise an amplifier binding domain and at least one imager binding domain, wherein the amplifier domain comprises a nucleotide complementary to a nucleotide sequence of an adapter binding domain, and wherein the imager strands comprising a detectable label and a nucleotide sequence that is complementary to a nucleotide sequence of an imager binding domain; (f) optionally removing unbound adapter strands and imager strands; and (g) imaging the sample to detect location of bound imager strands. In some embodiments, the method further comprises a step (h) of extinguishing a detectable signal from the detectable labels of the bound imager strands, and optionally, repeating steps (e)-(h) at least once with a second set of adaptor strands bound/hybridized with imager strands and having a unique nucleotide sequence relative to the nucleotide sequences of the adaptor strands in the first set. In some embodiments, steps (e)-(j) can be repeated once or multiple times. For example, steps (e)-(j) can be repeated 1-10 times or more. In some embodiments, steps (e)-(j) are repeated 1, 2, 3, 4, 5, 6, 7, 8, 9 or 10 times.

In some embodiments of any one of the aspects described herein, the adaptor strands and the imager strands are mixed together prior to contacting with the sample. Thus, in some embodiments, the method comprises: (a) contacting a sample being tested for the presence of one or more targets with one or more target-specific target-binding molecules, wherein each target-binding molecule is linked to a barcode strand, and wherein target-binding molecules of different specificity are linked to different barcode strands; (b) optionally removing unbound target-binding molecules; (c) contacting the sample with amplifier strands comprising a barcode binding domain and a plurality of adapter binding domains, wherein the barcode binding domain comprises a nucleotide sequence that is substantially complementary to a barcode strand; (d) optionally removing unbound amplifier strands; (e) contacting the sample with a first set of adaptor strands bound/hybridized with imager strands, wherein the adaptor strands comprise an amplifier binding domain and at least one imager binding domain, wherein the amplifier domain comprises a nucleotide complementary to a nucleotide sequence of an adapter binding domain, wherein the imager strands comprise a detectable label and a nucleotide sequence that is complementary to a nucleotide sequence of an imager binding domain of the adaptor strand; (f) optionally removing any unbound adapter and imager strands; and (g) imaging the sample to detect location of bound imager strands.

In some embodiments, the method comprises: (a) contacting a sample being tested for the presence of one or more targets with one or more target-specific target-binding molecules, wherein each target-binding molecule is linked to a barcode strand, and wherein target-binding molecules of different specificity are linked to different barcode strands; (b) optionally removing unbound target-binding molecules; (c) contacting the sample with amplifier strands comprising a barcode binding domain and a plurality of adapter binding domains, wherein the barcode binding domain comprises a nucleotide sequence that is substantially complementary to a barcode strand; (d) optionally removing unbound amplifier strands; (e) contacting the sample with a first set of adaptor strands comprising an amplifier binding domain and at least one imager binding domain, wherein the amplifier domain comprises a nucleotide complementary to a nucleotide sequence of an adapter binding domain; (f) optionally removing unbound adapter strands; (g) contacting the sample with imager strands comprising a detectable label and a nucleotide sequence that is complementary to a nucleotide sequence of an imager binding domain; (h) optionally removing unbound imager strands; and (i) imaging the sample to detect location of bound imager strands.

In some embodiments, the method further comprises a step (j) of extinguishing signal from the detectable labels of the bound imager strands, and optionally, repeating steps (e)-(j) at least once with a second set of adaptor strands having a unique nucleotide sequence relative to the nucleotide sequences of the adaptor strands in the first set and bound/hybridized with imager strands. In some embodiments, steps (e)-(j) can be repeated once or multiple times. For example, steps (e)-(j) can be repeated 1-10 times or more. In some embodiments, steps (e)-(h) are repeated 1, 2, 3, 4, 5, 6, 7, 8, 9 or 10 times.

When the adaptor strands and the imager strands are mixed together prior to contacting with the sample, quencher strands, the reverse complements of the imager strands, can be added to the mixture to block the imager strands from binding aberrantly to the sample.

Imager strands that bind/hybridize with the adaptor strand are also referred to as “Universal Imager” herein.

The imager strands can bind/hybridize directly with the amplifier strand without the adaptor strands. Thus, in some embodiments, the method comprises: (a) contacting a sample being tested for the presence of one or more targets with one or more target-specific target-binding molecules, wherein each target-binding molecule is linked to a barcode strand, and wherein target-binding molecules of different specificity are linked to different barcode strands; (b) optionally removing unbound target-binding molecules; (c) contacting the sample with amplifier strands comprising a barcode binding domain, wherein the barcode binding domain comprises a nucleotide sequence that is substantially complementary to a barcode strand; (d) optionally removing unbound amplifier strands; (e) contacting the sample with a first set of imager strands comprising a detectable label and a nucleotide sequence that is substantially complementary to a nucleotide sequence of an imager binding domain; (f) optionally removing unbound imager strands; (g) imaging the sample to detect location of bound labeled imager strands; (h) extinguishing signal from the bound labeled imager strands; and (i) optionally, repeating steps (e)-(h), each time with second set of imager strands having a unique nucleotide sequence relative to the imager strands of the first set. In some embodiments, steps (e)-(h) can be repeated once or multiple times. For example, steps (e)-(h) can be repeated 1-10 times or more. In some embodiments, steps (e)-(h) are repeated 1, 2, 3, 4, 5, 6, 7, 8, 9 or 10 times.

### Adaptor Strands

Embodiments of the various aspects described herein include adaptor strands. Adaptor strands are nucleic acids that binds/hybridizes with an amplifier strand on one end and with an imager strand at the other end. Generally, an adaptor strand comprises an amplifier binding domain and an imager binding domain. The amplifier binding domain has a nucleotide sequence substantially complementary to a nucleotide sequence of an adaptor binding domain of an amplifier strand. The amplifier binding domain of the adaptor strand and the adaptor binding domain of amplifier strand have sufficient complementary for hybridization. An adaptor strand can be capable of binding to one or more identical imager strands, e.g., imager strands of identical sequence and comprising identical detectable label.

It is noted that the amplifier binding domain and the imager binding domain can be positioned in any desired ordered. For example, the amplifier binding domain can be at the 5′-end of the adaptor strand and the imager binding domain can be at the 3′-end of the adaptor strand, or the amplifier binding domain can be at the 3′-end of the adaptor strand and the imager binding domain can be at the 5′-end of the adaptor strand. Accordingly, in some embodiments of any one of the aspects described herein, the amplifier binding domain of the adaptor strand is at 5′ of the imager binding domain. In some other embodiments, the amplifier binding domain of the adaptor strand is at 3′ of the imager binding domain.

An adaptor strand can be from about 40 to about 100, or more nucleotides in length. For example, the adaptor strand can be from about 40 to about 75 nucleotides in length. In some embodiments, the adaptor strand can be 40, 41, 42, 43, 44, 45, 46, 47, 48, 49, 50, 50, 51, 52, 53, 54, 55, 56, 57, 58, 5, or 60 nucleotides in length. Generally, the adaptor strand is a single-stranded nucleic acid. However, when not hybridized to an amplifier strand, the adaptor strand can be at least partially double-stranded. For example, the adaptor strand can have a hair-pin structure.

Embodiments of the various aspects described herein include removing the adaptor strands from the amplifier strands. The amplifier binding domain and the imager binding domain of the adaptor strands are designed such that the adaptor strand can be removed from the amplifier strand without removing any imager strand bound/hybridized with the adaptor strand. This can be due to difference in lengths of the amplifier binding domain and the imager binding domain of the adaptor strand and/or their nucleotide sequences.

In some embodiments, the adaptor strand can be removed from the amplifier strand without removing any imager strand bound/hybridized with the adaptor strand due to difference in the melting temperatures. For example, a melting temperature of the adaptor strand binding/hybridizing with the amplifier strand is lower than a melting temperature of the adaptor strand binding/hybridizing with an imager strand. In some embodiments, the melting temperature of the adaptor strand binding/hybridizing with the amplifier strand is at least 5° C. lower than the melting temperature of the adaptor strand binding/hybridizing with an imager strand. For example, the melting temperature of the adaptor strand binding/hybridizing with the adaptor strand is at least 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. lower than the melting temperature of the adaptor strand binding/hybridizing with an imager strand. For example, the melting temperature of the adaptor strand binding/hybridizing with the adaptor strand is 8° C., 9° C., 10° C., 11° C., 12° C., 13° C., 14° C., 15° C., or 16° C. lower than the melting temperature of the adaptor strand binding/hybridizing with an imager strand.

Additionally, the amplifier binding domain of the adaptor strand and the barcode binding domain of the amplifier strand that the adaptor strand hybridizes with are designed such that the adaptor strand can be removed from the amplifier strand without removing the amplifier strand from the barcode strand it is hybridized with. This can be due to difference in lengths of the amplifier binding domain of the adaptor strand and the barcode binding domain of the amplifier strand and/or their nucleotide sequences.

In some embodiments, the adaptor strand can be removed from the amplifier strand without removing the amplifier strand from the barcode strand it is hybridized with the adaptor strand due to difference in the melting temperatures. For example, a melting temperature of the adaptor strand binding/hybridizing with the amplifier strand is lower than a melting temperature of the amplifier strand binding/hybridizing with a barcode strand. In some embodiments, the melting temperature of the adaptor strand binding/hybridizing with the amplifier strand is at least 5° C. lower than the melting temperature of the amplifier strand binding/hybridizing with a barcode strand. For example, the melting temperature of the adaptor strand binding/hybridizing with the adaptor strand is at least 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. lower than the melting temperature of the amplifier strand binding/hybridizing with a barcode strand. For example, the melting temperature of the adaptor strand binding/hybridizing with the adaptor strand is 8° C., 9° C., 10° C., 11° C., 12° C., 13° C., 14° C., 15° C., or 16° C. lower than the melting temperature of the amplifier strand binding/hybridizing with a barcode strand.

In some embodiments, an imager binding domain of the adaptor strand is from about 21 to about 50 or more nucleotides in length. For example, the imager binding domain of the adaptor strand is from about 21 to about 50 nucleotides in length. In some embodiments, the imager binding domain of the adaptor strand is 21, 22, 23, 24, 25, 26, 27, 28, 29, 30, 31, 32, 33, 34, 35, 36, 37, 38, 39, or 40 nucleotides in length. For example, the imager binding domain of the adaptor strand is 23, 24, 25, 26, 27, 28, 29 or 30 nucleotides in length. In some embodiments, the imager binding domain of the adaptor strand is 25, 26, 27, 28, 29 or 30 nucleotides in length.

The amplifier domain of the adaptor strand can be from about 10 to about 20 nucleotides in length. For example, the amplifier domain of the adaptor strand can be 11, 12, 13, 14, 15, 16, 17, 18, or 19 nucleotides in length. In some embodiments, the amplifier domain of the adaptor strand can be 15, 16, 17, or 18 nucleotides in length.

Generally, the length of the amplifier binding domain is less than the length of the imager binding domain. For example, the length of the amplifier binding domain is about 3 to about 20 or more nucleotides less than the length of the imager binding domain. In some embodiments, the length of the amplifier binding domain is 4, 5, 6, 7, 8, 9, 10, 11, 12, 13, 14, 15 or more nucleotides less than the length of the imager binding domain. For example, the length of the amplifier binding domain is 6, 7, 8, 9, or 10 nucleotides less than the length of the imager binding domain.

### Barcode Strands

Embodiments of the various aspects described herein include a barcode strand. Barcode strands are nucleic acids that are linked to target-binding molecules and hybridize with an amplifier strand. Generally, at least a part of the barcode strand comprises a nucleotide sequence substantially complementary to a nucleotide sequence of a barcode domain of an adaptor strand for hybridization.

A barcode strand can be from about 21 to about 50 or more nucleotides in length. For example, the barcode strand can be from about 21 to about 50 nucleotides in length. In some embodiments, the barcode strand is 21, 22, 23, 24, 25, 26, 27, 28, 29, 30, 31, 32, 33, 34, 35, 36, 37, 38, 39, or 40 nucleotides in length. For example, the barcode strand is 23, 24, 25, 26, 27, 28, 29 or 30 nucleotides in length. In some embodiments, the barcode strand is 25, 26, 27, 28, 29 or 30 nucleotides in length.

Generally, the barcode strand is a single-stranded nucleic acid. However, when not hybridized to an amplifier strand, the barcode strand can be at least partially double-stranded. For example, the barcode strand can have a hair-pin structure.

In some embodiments of any one of the aspects described herein, the barcode strand is capable of stably hybridizing to its complementary amplifier strand. Stable binding can be a result of the length of the barcode binding domain of the amplifier strand (and conversely the length of the barcode strand) or it can be the result of the particular conditions under which hybridization occurs (e.g., salt concentration, temperature, etc.).

A barcode strand can be linked directly to the target-binding molecule via a bond. In some embodiments, a barcode strand can be linked to the target-binding molecule via a linker. Further, a barcode strand can be linked to the target-binding molecule by its 5′-end or 3′-end. Accordingly, in some embodiments a barcode strand can be linked to the target-binding molecule by its 5′-end. In some other embodiments, a barcode strand can be linked to the target-binding molecule by its 3′-end.

### Amplifier Strands

Embodiments of the various aspects described herein include amplifier strands. Amplifier strands are nucleic acids that bind/hybridize with a barcode strand on one end and with a plurality of imager strands or adaptor strands on the other end. Generally, an amplifier strand comprises a barcode binding domain and a plurality of adaptor binding domains or imager binding domains. The barcode binding domain has a nucleotide sequence substantially complementary to a nucleotide sequence of at least part of a barcode strand. The barcode binding domain and the barcode strand have sufficient complementary for hybridization.

Generally, the barcode binding domain of the amplifier strand is from about 21 to about 50 or more nucleotides in length. For example, the barcode binding domain of the amplifier strand is from about 21 to about 50 nucleotides in length. In some embodiments, the barcode binding domain of the amplifier strand is 21, 22, 23, 24, 25, 26, 27, 28, 29, 30, 31, 32, 33, 34, 35, 36, 37, 38, 39, or 40 nucleotides in length. For example, the barcode binding domain of the amplifier strand is 23, 24, 25, 26, 27, 28, 29 or 30 nucleotides in length. In some embodiments, the barcode binding domain of the amplifier strand is 25, 26, 27, 28, 29 or 30nucleotides in length.

In some embodiments, the amplifier strand comprises a barcode binding domain and a plurality of adaptor binding domains. For example, the amplifier strand comprises a barcode binding domain and at least 5 or more adaptor binding domains. In some embodiments, the amplifier strand comprises at least 6, 7, 8, 9, 10, 11, 12, 13, 14, 15, 16, 17, 18, 19, 20 or more adaptor binding domains. For example, the amplifier strand comprises 8, 9, 10, 11, 12, 13, 14 or 15 adaptor binding domains. It is noted that an amplifier strand can be capable of binding to one or more identical adaptor strands, e.g., adaptor strands comprising amplifier binding domains of identical sequence and, optionally, comprising imager binding domains of identical sequence.

It is noted that the barcode binding domain and the adaptor binding domains can be positioned in any desired ordered. For example, the barcode binding domain can be at the 5′-end of the amplifier strand and the adaptor binding domains can be at the 3′-end of the amplifier strand, or the barcode binding domain can be at the 3′-end of the amplifier strand and the adaptor binding domains can be at the 5′-end of the amplifier strand. Accordingly, in some embodiments of any one of the aspects described herein, the barcode binding domain of the amplifier strand is at 5′ of the adaptor binding domains. In some other embodiments, the barcode binding domain of the amplifier strand is at 3′ of the adaptor binding domain.

Each adaptor binding domain of the amplifier strand can be independently from about 10 to about 20 nucleotides in length. For example, each adaptor binding domain of the amplifier strand can be independently 11, 12, 13, 14, 15, 16, 17, 18, or 19 nucleotides in length. In some embodiments, each adaptor binding domain of the amplifier strand can be independently 15, 16, 17, or 18 nucleotides in length. In some embodiments, all the adaptor binding domains of the amplifier strand are the same length. In some embodiments, all the adaptor binding domains of the amplifier strand are identical, e.g., comprise an identical nucleotide sequence.

Generally, the length of the adaptor binding domains is less than the length of the barcode binding domain. For example, the length of the adaptor binding domains is about 3 to about 20 or more nucleotides less than the length of the barcode binding domain. In some embodiments, the length of the barcode binding domains is 4, 5, 6, 7, 8, 9, 10, 11, 12, 13, 14, 15 or more nucleotides less than the length of the barcode binding domain. For example, the length of the adaptor binding domains is 6, 7, 8, 9, or 10 nucleotides less than the length of the barcode binding domain.

In some embodiments, the amplifier strand comprises a barcode binding domain and a plurality of imager binding domains. For example, the amplifier strand comprises a barcode binding domain and at least 5 or more imager binding domains. In some embodiments, the amplifier strand comprises at least 6, 7, 8, 9, 10, 11, 12, 13, 14, 15, 16, 17, 18, 19, 20 or more imager binding domains. For example, the amplifier strand comprises 8, 9, 10, 11, 12, 13, 14 or 15 imager binding domains. It is noted that an amplifier strand can be capable of binding to one or more identical imager strands, e.g., imager strands comprising identical sequence and detectable label.

It is noted that the barcode binding domain and the imager binding domains can be positioned in any desired ordered. For example, the barcode binding domain can be at the 5′-end of the amplifier strand and the imager binding domains can be at the 3′-end of the amplifier strand, or the barcode binding domain can be at the 3′-end of the amplifier strand and the imager binding domains can be at the 5′-end of the amplifier strand. Accordingly, in some embodiments of any one of the aspects described herein, the barcode binding domain of the amplifier strand is at 5′ of the imager binding domains. In some other embodiments, the barcode binding domain of the amplifier strand is at 3′ of the imager binding domain.

Each imager binding domain of the amplifier strand can be independently from about 10 to about 20 nucleotides in length. For example, each imager binding domain of the amplifier strand can be independently 11, 12, 13, 14, 15, 16, 17, 18, or 19 nucleotides in length. In some embodiments, each imager binding domain of the amplifier strand can be independently 15, 16, 17, or 18 nucleotides in length. In some embodiments, all the imager binding domains of the amplifier strand are the same length. In some embodiments, all the imager binding domains of the amplifier strand are identical, e.g., comprise an identical nucleotide sequence.

Generally, the length of the imager binding domains is less than the length of the barcode binding domain. For example, the length of the imager binding domains is about 3 to about 20 or more nucleotides less than the length of the barcode binding domain. In some embodiments, the length of the imager binding domains is 4, 5, 6, 7, 8, 9, 10, 11, 12, 13, 14, 15 or more nucleotides less than the length of the barcode binding domain. For example, the length of the imager binding domains is 6, 7, 8, 9, or 10 nucleotides less than the length of the barcode binding domain.

An amplifier strand can be from about 150 to about 500, or more nucleotides in length. For example, the amplifier strand can be from about 175 to about 250 nucleotides in length. In some embodiments, the amplifier strand can be about 175, about 180, about 185, about 190, about 195, about 200, about 205, about 210, about 215, about 220, or about 225 nucleotides in length.

Generally, the amplifier strand is single-stranded nucleic acid. However, when not hybridized to a barcode strand, an adaptor strand or an imager strand, the amplifier strand can be at least partially double-stranded. For example, the amplifier strand can have a hair-pin structure.

### Imager Strands

Embodiments of the various aspects described herein include imager strands. Imager strands are nucleic acids that bind/hybridize with an adaptor strand or an amplifier and comprise a detectable label. Generally, an imager strand comprises a nucleotide sequence substantially complementary to an imager binding domain of an adaptor strand or an amplifier strand. The imager strand and the imager binding domain of the adaptor strand or the amplifier strand have sufficient complementary for hybridization.

It is noted that the detectable label can be positioned at any position of the imager strand. For example, the detectable label can be at the 5′-end of the imager strand. Conversely, the detectable label can be at the 3′-end of the imager strand.

In some embodiments, at least first two nucleotides at the end to imager strand that is linked to the detectable label do not hybridized with the strand the imager strand binds or hybridizes with. For example, the imager strand comprises, at its end the detectable label is attached to, at least 2 (e.g., 3, 4, 5, 6, 7, 8, 9, 10 or more) nucleotides that do not hybridized with the strand the imager strand hybridizes with.

In some embodiments of any one of the aspects described herein, an imager strand comprises two or more detectable labels. For example, the imager strand comprises a detectable label at the 5′-end and a detectable label at the 3′-end. When an imager strand comprises two or more detectable labels, they can be same or different. In some embodiments, the imager strand comprises two or more identical detectable labels.

Each imager strand can be independently from about 10 to about 60 nucleotides, or more, in length, including 10, 15, 20, 25 30, 35, 40, 45, 50, 55 or 60 nucleotides in length. For example, imager strands comprising a nucleotide sequence substantially complementary to a nucleotide sequence of an imager binding domain of an amplifier strand can be independently from about 10 to about 20 nucleotides in length. In some embodiments, each imager strand comprising a nucleotide sequence substantially complementary to a nucleotide sequence of an imager binding domain of an amplifier strand can be independently 11, 12, 13, 14, 15, 16, 17, 18, or 19 nucleotides in length. For example, each imager strand can be independently 15, 16, 17, or 18 nucleotides in length.

Imager strands comprising a nucleotide sequence substantially complementary to a nucleotide sequence of an imager binding domain of an adaptor strand can be independently from about 21 to about 50 nucleotides in length. In some embodiments, imager strands comprising a nucleotide sequence substantially complementary to a nucleotide sequence of an imager binding domain of an adaptor strand can be independently 21, 22, 23, 24, 25, 26, 27, 28, 29, 30, 31, 32, 33, 34, 35, 36, 37, 38, 39, or 40 nucleotides in length. For example, imager strands comprising a nucleotide sequence substantially complementary to a nucleotide sequence of an imager binding domain of an adaptor strand can be independently 23, 24, 25, 26, 27, 28, 29 or 30 nucleotides in length. In some embodiments, imager strands comprising a nucleotide sequence substantially complementary to a nucleotide sequence of an imager binding domain of an adaptor strand can be independently 25, 26, 27, 28, 29 or 30 nucleotides in length.

Generally, the imager strand is single-stranded nucleic acid. However, when not hybridized to an amplifier strand or an adaptor strand, the imager strand can be at least partially double-stranded. For example, the imager strand can have a hair-pin structure.

A detectable label can be linked to the imager strand via a cleavable linker. Generally, the cleavable linker comprises at least one cleavable linking group. A cleavable linking group is one which is sufficiently stable but which can be cleaved to release the two parts the linker is holding together. Cleavable linking groups are susceptible to light or cleavage agents, e.g., pH, redox potential or the presence of degradative molecules. Examples of such degradative agents include: redox agents which are selected for particular substrates or which have no substrate specificity, including, e.g., oxidative or reductive enzymes or reductive agents such as mercaptans that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

In some embodiments, the cleavable linker can be selected from the group consisting of photocleavable linkers, hydrolyzable linkers, redox cleavable linkers, phosphate-based cleavable linkers, acid cleavable linkers, ester-based cleavable linkers, peptide-based cleavable linkers, and any combinations thereof. In some embodiments, the cleavable linker can comprise a disulfide bond, a tetrazine-trans-cyclooctene group, a sulfhydryl group, a nitrobenzyl group, a nitroindoline group, a bromo hydroxycoumarin group, a bromo hydroxyquinoline group, a hydroxyphenacyl group, a dimethoxybenzoin group, or any combinations thereof.

Any art-recognized photocleavable linker can be used. In some embodiments, the cleavable linker can comprise a photocleavable linker. Generally, photocleavable linkers contain a photolabile functional group that is cleavable upon exposure to a light source (e.g., UV light) or specific wavelength. Non-limiting examples of photocleavable spacers can be found, for example, in U.S. Pat. Nos. 6,589,736 B1; 7,622,279 B2; 9,371,348 B2; 7,547,530 B2; and 7,057,031 B2; and PCT Publication No. WO2014200767, contents of all of which are incorporated herein by reference in their entirety.

In some embodiments, the detectable label can be linked to the imager strand via a linker that can be cleaved chemically. One class of chemically cleavable linking groups is redox cleavable linking groups, which can be used according to the present invention that are cleaved upon reduction or oxidation. An example of reductively cleavable linking group is a disulfide linking group (—S—S—). For example, the linker comprises one or more disulfide (S—S) bonds. Linker comprising disulfide bonds can be cleaved by use of a reducing agent (e.g., tris(2-carboxyethyl)phosphine, dithiothreitol (DTT), or other reducing agent using reagents know in the art). A disulfide bond also can be susceptible to pH.

In some embodiments, the cleavable linker can comprise an acid cleavable linking group. Acid cleavable linking groups are linking groups that are cleaved under acidic conditions. Generally, acid cleavable linking groups are cleaved in an acidic environment with a pH of about 6.5 or lower (e.g., about 6.0, 5.5, 5.0, or lower), or by agents such as enzymes that can act as a general acid. Examples of acid cleavable linking groups include but are not limited to hydrazones, esters, and esters of amino acids. Acid cleavable groups can have the general formula —C═NNH—, —C(O)O—, or —OC(O)—.

In some embodiments of any one of the aspects described herein, the detectable label can be linked to the imager strand via a photo-cleavable linker. Exemplary photo-cleavable linkers include, but are not limited to, linkers containing o-nitrobenzyl, p-nitrobenzyl, m-nitrobenzyl, desyl, trans-cinnamoyl, m-nitrophenyl, benzylsulfonyl, nitoindoline, bromohydroxycoumarin, bromohydroxyquinoline, and/or hydroxyphena groups. In some embodiments, a photo-cleavable linker can be cleaved by exposure to ultraviolet light.

In some embodiments, the detectable label can be linked to the imager strand via a linker that can be cleaved enzymatically, i.e., enzyme-cleavable linker. Exemplary enzyme cleavable linkers include, but are not limited to, DNA, RNA, peptide linkers, β-glucuronide linkers, or any combinations thereof.

In some embodiments, the imager strand can be self-quenching, intending that the unbound imager nucleic acid can carry a quencher moiety that is in close proximity with the detectable label. To achieve this, the imager strand can be designed to adopt either a molecular beacon-like structure, a hair-pin structure or a hemiduplex structure. Without wishing to be bound by a theory, this self-quenching variation can be used to reduce background and/or avoid the washing step. Additionally, or alternatively, the binding and imaging buffer can contain additives routinely used in FISH, Northern Blotting and Southern Blotting (e.g., negatively charged polymers such as dextran sulfate and heparin) to reduce non-specific binding.

### Blocker Strands

Embodiments of the various aspects described herein include blocker strands. Blocker strands are nucleic acid strands that bind/hybridize with an adaptor strand or an amplifier. Generally, blocker strands are identical, e.g., comprise an identical nucleotide sequence, to the imager strands, but blocker strands do not comprise a detectable label.

### Target-Binding Molecules

Embodiments of the various aspects described herein include target binding molecules. The terms “target binding molecule” and “target binding ligand” are used interchangeably herein and refer to a molecule that binds to or interacts with a target molecule. In other words, a target binding ligand or molecule is a molecule that is capable of binding with a target molecule. The targeting binding ligand can be a natural or synthetic molecule (e.g., a molecular receptor) that binds to a target molecule. Exemplary target binding ligands include, but are not limited to, an antibody, an antigen binding fragment of an antibody, a nucleic acid, a receptor, a ligand for a receptor, an antigen, or an enzyme. The target binding ligand is also referred to as a “capture agent” or “capture molecule” herein.

In some embodiments of any one of the aspects described herein, the binding of the target binding ligand to the target molecule is a specific binding such that it is selective to that target above non-targets. For example the dissociation constant between the target binding ligand and target molecule is at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain embodiments, the specific binding refers to binding where the target binding ligand binds to its target molecule without substantially binding to any other species in the sample/test solution.

By way of non-limiting examples, a target binding ligand can be selected from antibodies, adnectins, ankyrins, other antibody mimetics and other protein scaffolds, aptamers, nucleic acid (e.g., an RNA or DNA aptamer), protein, peptide, binding partner, oligosaccharides, polysaccharides, lipopolysaccharides, cellular metabolites, cells, viruses, subcellular particles, haptens, pharmacologically active substances, alkaloids, steroids, vitamins, amino acids, avimers, peptidomimetics, hormone receptors, cytokine receptors, synthetic receptors, sugars or molecularly imprinted polymer. The target binding ligand can be selective to a specific target or class of targets such as toxins and biomolecules. For example, the target can be ions, molecules, oligomers, polymers, proteins, peptides, nucleic acids, toxins, biological threat agents such as spore, viral, cellular and protein toxins, carbohydrates (e.g., mono saccharides, disaccharides, oligosaccharides, polyols, and polysaccharides) and combinations of these (e.g., copolymers including these).

In some embodiments of any one of the aspects described herein, the target binding molecule is an antibody or antigen binding fragment thereof. As used herein, the terms “antibody” and “antibodies” include polyclonal antibodies, monoclonal antibodies, humanized or chimeric antibodies, single chain Fv antibody fragments, Fab fragments, and F(ab)₂ fragments. Antibodies having specific binding affinity for a target of interest (e.g., an antigen) can be produced through standard methods. As used herein, the terms “antibody” and “antibodies” refer to intact antibody, or a binding fragment thereof that competes with the intact antibody for specific binding and includes chimeric, humanized, fully human, and bispecific antibodies.

In some embodiments, the target binding molecule is a nucleic acid. For example, the target binding molecule is an aptamer. In some embodiments, a target is a nucleic acid and the target-binding molecule is a nucleic acid strand comprising a nucleotide sequence substantially complementary to at least a portion of the target nucleic acid.

### Targets

Embodiments of the various aspects described herein include target s. A “target” is any moiety that one wishes to observe or quantitate and for which a target-binding molecule exists. A target, in some embodiments, can be non-naturally occurring. The target, in some embodiments, can be a biomolecule. As used herein, a “biomolecule” is any molecule that is produced by a living organism, including large macromolecules such as proteins, polysaccharides, lipids and nucleic acids (e.g., DNA and RNA such as mRNA), as well as small molecules such as primary metabolites, secondary metabolites, and natural products. Examples of biomolecules include, without limitation, DNA, RNA, cDNA, or the DNA product of RNA subjected to reverse transcription, A23187 (Calcimycin, Calcium Ionophore), Abamectine, Abietic acid, Acetic acid, Acetylcholine, Actin, Actinomycin D, Adenosine, Adenosine diphosphate (ADP), Adenosine monophosphate (AMP), Adenosine triphosphate (ATP), Adenylate cyclase, Adonitol, Adrenaline, epinephrine, Adrenocorticotropic hormone (ACTH), Aequorin, Aflatoxin, Agar, Alamethicin, Alanine, Albumins, Aldosterone, Aleurone, Alpha-amanitin, Allantoin, Allethrin, α-Amanatin, Amino acid, Amylase, Anabolic steroid, Anethole, Angiotensinogen, Anisomycin, Antidiuretic hormone (ADH), Arabinose, Arginine, Ascomycin, Ascorbic acid (vitamin C), Asparagine, Aspartic acid, Asymmetric dimethylarginine, Atrial-natriuretic peptide (ANP), Auxin, Avidin, Azadirachtin A—C35H44O16, Bacteriocin, Beauvericin, Bicuculline, Bilirubin, Biopolymer, Biotin (Vitamin H), Brefeldin A, Brassinolide, Brucine, Cadaverine, Caffeine, Calciferol (Vitamin D), Calcitonin, Calmodulin, Calmodulin, Calreticulin, Camphor—(C10H16O), Cannabinol, Capsaicin, Carbohydrase, Carbohydrate, Carnitine, Carrageenan, Casein, Caspase, Cellulase, Cellulose—(C6H10O5), Cerulenin, Cetrimonium bromide (Cetrimide)—C19H42BrN, Chelerythrine, Chromomycin A3, Chaparonin, Chitin, α-Chloralose, Chlorophyll, Cholecystokinin (CCK), Cholesterol, Choline, Chondroitin sulfate, Cinnamaldehyde, Citral, Citric acid, Citrinin, Citronellal, Citronellol, Citrulline, Cobalamin (vitamin B12), Coenzyme, Coenzyme Q, Colchicine, Collagen, Conline, Corticosteroid, Corticosterone, Corticotropin-releasing hormone (CRH), Cortisol, Creatine, Creatine kinase, Crystallin, α-Cyclodextrin, Cyclodextrin glycosyltransferase, Cyclopamine, Cyclopiazonic acid, Cysteine, Cystine, Cytidine, Cytochalasin, Cytochalasin E, Cytochrome, Cytochrome C, Cytochrome c oxidase, Cytochrome c peroxidase, Cytokine, Cytosine—C4H5N3O, Deoxycholic acid, DON (DeoxyNivalenol), Deoxyribofuranose, Deoxyribose, Deoxyribose nucleic acid (DNA), Dextran, Dextrin, DNA, Dopamine, Enzyme, Ephedrine, Epinephrine—C9H13NO3, Erucic acid—CH3(CH2)77CH═CH(CH2)11COOH, Erythritol, Erythropoietin (EPO), Estradiol, Eugenol, Fatty acid, Fibrin, Fibronectin, Folic acid (Vitamin M), Follicle stimulating hormone (FSH), Formaldehyde, Formic acid, Formnoci, Fructose, Fumonisin B1, Gamma globulin, Galactose, Gamma globulin, Gamma-aminobutyric acid, Gamma-butyrolactone, Gamma-hydroxybutyrate (GHB), Gastrin, Gelatin, Geraniol, Globulin, Glucagon, Glucosamine, Glucose—C6H12O6, Glucose oxidase, Gluten, Glutamic acid, Glutamine, Glutathione, Gluten, Glycerin (glycerol), Glycine, Glycogen, Glycolic acid, Glycoprotein, Gonadotropin-releasing hormone (GnRH), Granzyme, Green fluorescent protein, Growth hormone, Growth hormone-releasing hormone (GHRH), GTPase, Guanine, Guanosine, Guanosine triphosphate (+GTP), Haptoglobin, Hematoxylin, Heme, Hemerythrin, Hemocyanin, Hemoglobin, Hemoprotein, Heparan sulfate, High density lipoprotein, HDL, Histamine, Histidine, Histone, Histone methyltransferase, HLA antigen, Homocysteine, Hormone, human chorionic gonadotropin (hCG), Human growth hormone, Hyaluronate, Hyaluronidase, Hydrogen peroxide, 5-Hydroxymethylcytosine, Hydroxyproline, S-Hydroxytryptamine, Indigo dye, Indole, Inosine, Inositol, Insulin, Insulin-like growth factor, Integral membrane protein, Integrase, Integrin, Intein, Interferon, Inulin, Ionomycin, Ionone, Isoleucine, Iron-sulfur cluster, K252a, K252b, KT5720, KT5823, Keratin, Kinase, Lactase, Lactic acid, Lactose, Lanolin, Lauric acid, Leptin, Leptomycin B, Leucine, Lignin, Limonene, Linalool, Linoleic acid, Linolenic acid, Lipase, Lipid, Lipid anchored protein, Lipoamide, Lipoprotein, Low density lipoprotein, LDL, Luteinizing hormone (LH), Lycopene, Lysine, Lysozyme, Malic acid, Maltose, Melatonin, Membrane protein, Metalloprotein, Metallothionein, Methionine, Mimosine, Mithramycin A, Mitomycin C, Monomer, Mycophenolic acid, Myoglobin, Myosin, Natural phenols, Nucleic Acid, Ochratoxin A, Oestrogens, Oligopeptide, Oligomycin, Orcin, Orexin, Ornithine, Oxalic acid, Oxidase, Oxytocin, p53, PABA, Paclitaxel, Palmitic acid, Pantothenic acid (vitamin B5), parathyroid hormone (PTH), Paraprotein, Pardaxin, Parthenolide, Patulin, Paxilline, Penicillic acid, Penicillin, Penitrem A, Peptidase, Pepsin, Peptide, Perimycin, Peripheral membrane protein, Perosamine, Phenethylamine, Phenylalanine, Phosphagen, phosphatase, Phospholipid, Phenylalanine, Phytic acid, Plant hormones, Polypeptide, Polyphenols, Polysaccharides, Porphyrin, Prion, Progesterone, Prolactin (PRL), Proline, Propionic acid, Protamine, Protease, Protein, Proteinoid, Putrescine, Pyrethrin, Pyridoxine or pyridoxamine (Vitamin B6), Pyrrolysine, Pyruvic acid, Quinone, Radicicol, Raffinose, Renin, Retinene, Retinol (Vitamin A), Rhodopsin (visual purple), Riboflavin (vitamin B2), Ribofuranose, Ribose, Ribozyme, Ricin, RNA—Ribonucleic acid, RuBisCO, Safrole, Salicylaldehyde, Salicylic acid, Salvinorin-A—C23H28O8, Saponin, Secretin, Selenocysteine, Selenomethionine, Selenoprotein, Serine, Serine kinase, Serotonin, Skatole, Signal recognition particle, Somatostatin, Sorbic acid, Squalene, Staurosporin, Stearic acid, Sterigmatocystin, Sterol, Strychnine, Sucrose (sugar), Sugars (in general), superoxide, T2 Toxin, Tannic acid, Tannin, Tartaric acid, Taurine, Tetrodotoxin, Thaumatin, Topoisomerase, Tyrosine kinase, Taurine, Testosterone, Tetrahydrocannabinol (THC), Tetrodotoxin, Thapsigargin, Thaumatin, Thiamine (vitamin B1)—C12H17ClN4OS. HCl, Threonine, Thrombopoietin, Thymidine, Thymine, Triacsin C, Thyroid-stimulating hormone (TSH), Thyrotropin-releasing hormone (TRH), Thyroxine (T4), Tocopherol (Vitamin E), Topoisomerase, Triiodothyronine (T3), Transmembrane receptor, Trichostatin A, Trophic hormone, Trypsin, Tryptophan, Tubulin, Tunicamycin, Tyrosine, Ubiquitin, Uracil, Urea, Urease, Uric acid—C5H4N4O3, Uridine, Valine, Valinomycin, Vanabins, Vasopressin, Verruculogen, Vitamins (in general), Vitamin A (retinol), Vitamin B, Vitamin B1 (thiamine), Vitamin B2 (riboflavin), Vitamin B3 (niacin or nicotinic acid), Vitamin B4 (adenine), Vitamin B5 (pantothenic acid), Vitamin B6 (pyridoxine or pyridoxamine), Vitamin B12 (cobalamin), Vitamin C (ascorbic acid), Vitamin D (calciferol), Vitamin E (tocopherol), Vitamin F, Vitamin H (biotin), Vitamin K (naphthoquinone), Vitamin M (folic acid), Wortmannin and Xylose.

In some embodiments, a target can be a protein target such as, for example, proteins of a cellular environment (e.g., intracellular or membrane proteins). Examples of proteins include, without limitation, fibrous proteins such as cytoskeletal proteins (e.g., actin, arp2/3, coronin, dystrophin, FtsZ, keratin, myosin, nebulin, spectrin, tau, titin, tropomyosin, tubulin and collagen) and extracellular matrix proteins (e.g., collagen, elastin, f-spondin, pikachurin, and fibronectin); globular proteins such as plasma proteins (e.g., serum amyloid P component and serum albumin), coagulation factors (e.g., complement proteins, C1-inhibitor and C3-convertase, Factor VIII, Factor XIII, fibrin, Protein C, Protein S, Protein Z, Protein Z-related protease inhibitor, thrombin, Von Willebrand Factor) and acute phase proteins such as C-reactive protein; hemoproteins; cell adhesion proteins (e.g., cadherin, ependymin, integrin, Neam and selectin); transmembrane transport proteins (e.g., CFTR, glycophorin D and scramblase) such as ion channels (e.g., ligand-gated ion channels such nicotinic acetylcholine receptors and GABAa receptors, and voltage-gated ion channels such as potassium, calcium and sodium channels), synport/antiport proteins (e.g., glucose transporter); hormones and growth factors (e.g., epidermal growth factor (EGF), fibroblast growth factor (FGF), vascular endothelial growth factor (VEGF), peptide hormones such as insulin, insulin-like growth factor and oxytocin, and steroid hormones such as androgens, estrogens and progesterones); receptors such as transmembrane receptors (e.g. G-protein-coupled receptor, rhodopsin) and intracellular receptors (e.g., estrogen receptor); DNA-binding proteins (e.g., histones, protamines, CI protein); transcription regulators (e.g., c-myc, FOXP2, FOXP3, MyOD and P53); immune system proteins (e.g., immunoglobulins, major histocompatibility antigens and T cell receptors); nutrient storage/transport proteins (e.g., ferritin); chaperone proteins; and enzymes.

In some embodiments, target is a nucleic acid such as, for example, nucleic acids of a cellular environment. For example, target is a nucleic acid such as DNA or RNA. In some embodiments the target can be a genomic DNA, cDNA, RNA, the DNA product of RNA subjected to reverse transcription. In some embodiments, the target is a nucleic acid amplification product.

### Sample

In accordance with various embodiments described herein, a sample, including any specimen (processed or unprocessed) that is intended to be evaluated for the presence of one or more target molecules can be subjected to methods, compositions, kits and systems described herein. A “sample” can comprise cells (or a cell), tissue, or bodily fluid such as blood (serum and/or plasma), urine, semen, lymphatic fluid, cerebrospinal fluid or amniotic fluid. A sample can be obtained from (or derived from) any source including, without limitation, humans, animals, bacteria, viruses, microbes and plants. In some embodiments, a sample is a cell lysate or a tissue lysate. A sample can also contain mixtures of material from one source or different sources. A sample can be a spatial area or volume (e.g., a grid on an array, or a well in a plate or dish).

In some embodiments, the sample is dissociated cells that are immobilized to a solid surface (e.g. glass slide or cover slip), including individually immobilized. For example, the sample can be cells in blood. For example, the sample can contain cancer cells circulating in the blood (also known as circulating tumor cells, or CTCs). The sample can be cells grown in suspension. The sample can be cells disseminated from a solid tissue.

In some embodiments, the sample is pre-processed prior to contacting with the target-binding molecules.

It is noted that the methods described herein are not limited to detecting/imaging targets, e.g., biomolecule such as proteins and nucleic acids in situ. The method described herein can be used to detect/image targets in a variety of different samples. For example, the methods described herein can be used to detect/image target molecule in Western Blots, ELISA and the like.

### Detectable Label

Embodiments of the various aspects described herein include a detectable label. As used herein, the term “detectable label” refers to a molecule or composition capable of producing a detectable signal indicative of the presence of a target. Exemplary detectable labels include but are not limited to a fluorophore, a chemiluminescent label, colloidal gold, colored glass or plastic (e.g., polystyrene, polypropylene, and latex) beads, a radiolabel, a quantum dot, an enzyme, or any combination thereof.

In some embodiments of any of the aspects, a detectable label can be a fluorescent dye molecule, or fluorophore. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

In some embodiments of any of the aspects, a detectable label is selected from the group consisting of fluorescent molecules, radioisotopes, chromophores, chemiluminescent moieties, bioluminescent moieties, optical reporters, echogenic substances, non-metallic isotopes, paramagnetic metal ions, and ferromagnetic metals.

In some embodiments, a detectable label is selected from the group consisting of 5-Carboxyfluorescein (5-FAM); 1,5 IAEDANS; 1,8-ANS; 4-Methylumbelliferone; 5-carboxy-2,7-dichlorofluorescein; 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GFF; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Factamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FF ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP-Cyan Fluorescent Protein; Chlorophyll; Chromomycin A; Chromomycin A; CMFDA; Coelenterazine ; Coelenterazine cp; Coelenterazine f; Coelenterazine fcp; Coelenterazine h; Coelenterazine hep; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; EFF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; FaserPro; Faurodan; EDS 751; Feucophor PAF; Feucophor SF; FeucophorWS; Fissamine Rhodamine; Fissamine Rhodamine B; FOFO-1; FO-PRO-1; Fucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKF; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufm; RH 414; Rhod-2; Rhodamine; Rhodamine 110; Rhodamine 123; Rhodamine 5 GFD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65F; S65T; Sapphire GFP; Serotonin; Sevron Brilliant Red 2B; Sevron Brilliant Red 4G; Sevron Brilliant Red B; Sevron Orange; Sevron Yellow F; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XF665; X-Rhodamine; XRITC; Xylene Orange; Y66F; Y66H; Y66W; Yellow GFP; YFP; YO-PRO-1; YO-PRO-3; YOYO-1; and YOYO-3.

### Extinguishing a Detectable Signal

Embodiments of the various aspects described herein include a step of extinguish a detectable signal produced by a detectable label. By extinguishing a detectable signal is meant inactivating a detectable signal produced by the detectable label. This can be accomplished by removing the imager strands from the amplifier or adaptor strands they are hybridized to, by removing the adaptor strands from the amplifier strands they are hybridized to, by removing the detectable label from the bound imager strands, and/or or by modifying the detectable label.

It is noted that a strand can be removed from a complementary strand by altering temperature, by altering buffer conditions, addition of a complementary nucleic acid strand, and/or cleaving, modifying or degrading the strand to be removed. Exemplary means for removing a strand include but are not limited to increasing temperature; decreasing the concentration of counter-ions (e.g., free Mg++); introducing or increasing the concentration of denaturants (e.g. formamide, urea, DMSO and the like); chemically, photochemically or enzymatically cleaving, modifying or degrading the strand to be removed; or any combination thereof.

In some embodiments, the step of extinguish a detectable signal produced by a detectable label comprises addition of addition of a denaturant. For example, addition of formamide, urea, DMSO, or a combination thereof.

In some embodiments, the step of extinguish a detectable signal produced by a detectable label comprises decreasing the concentration of counter-ions (e.g., free Mg++).

In some embodiments, the step of extinguish a detectable signal produced by a detectable label comprises adding complementary nucleic acid strands, wherein complementary the nucleic acid strands comprise a nucleotide sequence substantially complementary to the strand to be removed or its complement.

In some embodiments of any one of the aspects described herein, the removal of the detectable label can be achieved by cleaving a linker between the imager strand and the detectable label.

In some embodiments, inactivation of the detectable label can be achieved by chemically or photochemically modifying the detectable signal produced by the detectable label. For example, when the detectable label is a fluorophore, it can be bleached by chemical agents (such as for example hydrogen peroxide or photobleached. As will be understood in the art, “photobleaching” refers to the photochemical alteration of a dye or a fluorophore molecule such that it is unable to fluoresce. This is caused by cleavage of covalent bonds or non-specific reactions between the fluorophore and surrounding molecules. Loss of activity caused by photobleaching can be controlled, in some embodiments, by reducing the intensity or time-span of light exposure, by increasing the concentration of fluorophores, by reducing the frequency and thus the photon energy of the input light, or by employing more robust fluorophores that are less prone to bleaching. Thus, photobleaching can be used to remove, modify or in some instance extinguish a detectable signal from a detectable label. Photobleaching can be performed by exposing the detectable label, e.g., fluorophore to a wavelength of light of suitable wavelength, energy and duration to permanently and irreversibly extinguish the ability of the fluorophore to emit further signal.

In some embodiments, the step of extinguish a detectable signal produced by a detectable label comprises removing and/or modifying the detectable label without removing the entirety of the imager strand from the amplifier or adaptor strand it is hybridized to.

In some embodiments, the step of extinguish a detectable signal produced by a detectable label comprises removing and/or modifying the detectable label without removing the entirety of the adaptor strand from the amplifier or adaptor strand it is hybridized to.

In some embodiments of any one of the aspects described herein, the strand to be removed, e.g., the adaptor strand or the imager strand comprises a deoxyuridine. The strand can be cleaved by uracil-DNA glycosylase. After cleavage, the binding strength of the strand is weakened.

### Domains

In some embodiments, a domain can independently utilize a 1-letter, 2-letter, 3-letter or 4-letter code. As used herein, a “1-letter code” means the domain only comprises only one type of nucleobase, i.e., only one of adenine, thymine/uracil, guanine, and cytosine, or modified versions thereof. For example, a domain utilizing a 1-letter code comprises a stretch of nucleotides comprising the same nucleobase or a modified version of the nucleobase. For example, a domain can comprise a stretch of poly A, polyT, polyC or polyG. A “2-letter code” means the domain only comprises two of the four nucleobases, i.e., only two of adenine, thymine/uracil, guanine, and cytosine, or modified versions thereof. For example, a 2-letter code can comprise or consist of nucleobases selected from the group consisting of adenine and thymine/uracil, adenine and guanine, adenine and cytosine, thymine/uracil and guanine, thymine/uracil and cytosine, and guanine and cytosine. A “3-letter code” means the domain comprises only three of the four nucleobases, i.e., only three of adenine, thymine/uracil, guanine, and cytosine, or modified versions thereof. For example, a 3-letter code can comprise or consists of nucleobases selected from the group consisting of: adenine, thymine/uracil, and guanine; adenine, thymine/uracil, and cytosine; adenine, guanine, and cytosine; and thymine/uracil, guanine, and cytosine.

In some embodiments of any one of the aspects described herein, at least one domain utilizes a 3-letter code. In some embodiments of any one of the aspects described herein, each domain independently utilizes a 3-letter code.

In some embodiments, at least one domain comprises same types of nucleobases. For example, a domain only comprises purine nucleobases or pyrimidine nucleobases.

It is noted that a two domains in a strand can be next to each other or they can be spaced apart be presence of one or more nucleotides between them.

### Nucleic Acid Modifications

A nucleic acid strand described herein can comprise a nucleic acid modification. For example, at least one of barcode strand, amplifier strand, adaptor strand and imager strand can independently comprise a nucleic acid modification. Exemplary nucleic acid modifications include, but are not limited to, nucleobase modifications, sugar modifications, inter-sugar linkage modifications, conjugates (e. g .., ligands), and any combinations thereof. Nucleic acid modifications also include unnatural, or degenerate nucleobases.

Exemplary modified nucleobases include, but are not limited to, inosine, xanthine, hypoxanthine, nubularine, isoguanosine, tubercidin, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

In some embodiments, a modified nucleobase can be selected from the group consisting of: inosine, xanthine, hypoxanthine, nubularine, isoguanosine, tubercidin, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl) guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 5-ethynyl-2′-deoxyuridine, 2-(thio)uracil,5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2-(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2-(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2-(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidin, isoguanosine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

Exemplary sugar modifications include, but are not limited to, 2′-Fluoro, 3′-Fluoro, 2′-OMe, 3′-OMe, 2′-deoxy modifications, and acyclic nucleotides, e.g., peptide nucleic acids (PNA), unlocked nucleic acids (UNA) or glycol nucleic acid (GNA).

In some embodiments, a nucleic acid modification can include replacement or modification of an inter-sugar linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2—N(CH3)—N(CH3)—), amide-3 (3′—CH₂—C(═O)—N(H)—5′) and amide-4(3′—CH₂—N(H)—C(═O)—5′), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′—S—CH₂—O—5′), formacetal (3′—O—CH₂—O—5′), oxime, methyleneimino, methylenecarbonylamino, methylenemethylimino (MMI, 3′—CH₂—N(CH₃)—O—5′), methylenehydrazo, methylenedimethylhydrazo, methyleneoxymethylimino, ethers (C3′—O—C5′), thioethers (C3′—S—C5′), thioacetamido (C3′—N(H)—C(═O)—CH₂—S—C5′, C3′—O—P(O)—O—SS—C5′, C3′—CH₂—NH—NH—C5′, 3′—NHP(O)(OCH₃)—O—5′ and 3′—NHP(O)(OCH₃)—O—5′).

In some embodiments, nucleic acid modifications can include peptide nucleic acids (PNA), bridged nucleic acids (BNA), morpholinos, locked nucleic acids (LNA), glycol nucleic acids (GNA), threose nucleic acids (TNA), or any other xeno nucleic acids (XNA) described in the art.

### Compositions

In some embodiments, the composition comprises: (i) a first target-specific target binding molecule linked to a first barcode strand; (ii) a first amplifier strand comprising a barcode binding domain and a plurality of adapter binding domains, wherein the barcode binding domain comprises a nucleotide sequence that is substantially is substantially complementary to the first barcode strand; (iii) at least one first adaptor strand comprising an amplifier binding domain and at least one imager binding domain, wherein the amplifier domain comprises a nucleotide complementary to a nucleotide sequence of the adapter binding domain of the first amplifier strand; and (iv) at least one first imager strand comprising a first detectable label and a nucleotide sequence that is complementary to a nucleotide sequence of the imager binding domain of the first adaptor strand. In some embodiments, the composition further comprises (i) a second target-specific target binding molecule linked to a second barcode strand, wherein the second target-binding molecule binds to a target that is different from the first target-binding molecule; (ii) a second amplifier strand comprising a barcode binding domain and a plurality of adapter binding domains, wherein the barcode binding domain comprises a nucleotide sequence that is substantially is substantially complementary to the second barcode strand; (iii) at least one second adaptor strand comprising an amplifier binding domain and at least one imager binding domain, wherein the amplifier domain comprises a nucleotide complementary to a nucleotide sequence of the adapter binding domain of the second amplifier strand; and (iv) at least one second imager strand comprising a second detectable label and a nucleotide sequence that is complementary to a nucleotide sequence of the imager binding domain of the second adaptor strand, and wherein the first and second detectable labels are different.

In some embodiments, the composition comprises: (i) a first target-binding molecule linked to a first barcode strand; (ii) a first amplifier strands comprising a barcode binding domain, wherein the barcode binding domain comprises a nucleotide sequence that is substantially complementary to the first barcode strand; and (iii) a first imager strand comprising a first detectable label and a nucleotide sequence that is substantially complementary to a nucleotide sequence of the first imager binding domain. In some embodiments, the composition further comprises: (i) a second target-binding molecule linked to a second barcode strand, wherein the second target-binding molecule binds to a target that is different from the first target-binding molecule; (ii) a second amplifier strands comprising a barcode binding domain, wherein the barcode binding domain comprises a nucleotide sequence that is substantially complementary to the second barcode strand; and (iii) a second imager strand comprising a second detectable label and a nucleotide sequence that is substantially complementary to a nucleotide sequence of the second imager binding domain, and wherein the first and second detectable labels are different.

In some embodiments of the composition, a melting temperature of the imager strand binding/hybridizing with the adaptor strand is lower than a melting temperature of the amplifier strand binding/hybridizing with the barcode strand and the imager strand binding/hybridizing with the adaptor strand. For example, a melting temperature of imager strand binding/hybridizing with the adaptor strand is at least about 5° C. lower than a melting temperature of the amplifier strand binding/hybridizing with the barcode strand and the imager strand binding/hybridizing with the adaptor strand.

In some embodiments of the composition, the amplifier binding domain of the adaptor strand is at 5′ of the imager binding domain. In some other embodiments of the composition, the amplifier binding domain of the adaptor strand is at 3′ of the imager binding domain.

In some embodiments of the composition, the barcode binding domain of the amplifier strand is 5′ of the plurality of adapter binding domains. In some other embodiments of the composition, the barcode binding domain of the amplifier strand is 3′ of the plurality of adapter binding domains.

In some embodiments of the composition, a melting temperature of the imager strand binding/hybridizing with the adaptor strand is from about 35° C. to about 45° C.

In some embodiments of the composition, a melting temperature of the adaptor strand binding/hybridizing with the amplifier strand is at least 50° C. or higher.

In some embodiments of the composition, the amplifier strand comprises at least 5 (e.g., 5, 6, 7, 8, 9, 10, 11, 12, 13, 14, 15 or more) adaptor binding domains. For example, the amplifier strand comprises 10 adaptor binding domains.

In some embodiments of the composition, the imager strand is hybridized with the adaptor strand. In some embodiments of the composition, the adaptor strand is hybridized with the amplifier strand. In some embodiments of the composition, the imager strand is hybridized with the adaptor strand and the adaptor strand is hybridized with the amplifier strand.

In some embodiments of the composition, a melting temperature of imager strand binding/hybridizing with the amplifier strand is lower than the amplifier strand binding/hybridizing with the barcode strand. In some other embodiments of the composition, a melting temperature of imager strand binding/hybridizing with the amplifier strand is at least about 5° C. lower than the amplifier strand binding/hybridizing with the barcode strand.

In some embodiments of the composition, the barcode binding domain of the amplifier strand is 5′ of the plurality of imager binding domains. In some other embodiments, the barcode binding domain of the amplifier strand is 3′ of the plurality of imager binding domains.

In some embodiments of the composition, a melting temperature of the imager strand binding/hybridizing with the amplifier strand is from about 35° C. to about 45° C.

In some embodiments of the composition, the amplifier strand comprises at least 5 (e.g., 5, 6, 7, 8, 9, 10, 11, 12, 13, 14, 15 or more) imager binding domains.

In some embodiments of the composition, the imager strand is hybridized with the amplifier strand.

In some embodiments of the composition, a melting temperature of the amplifier strand binding/hybridizing with the barcode strand is at least 50° C. or higher.

In some embodiments of the composition, the detectable label is attached at the 5′-end of the imager strand it is attached to.

In some embodiments of the composition, detectable label is attached at the 3′-end of the imager strand it is attached to.

In some embodiments of the composition, the imager strand comprises, at its end the detectable label is attached to, at least two nucleotides that do not hybridized with the strand the imager strand is bound/hybridized to.

In some embodiments of the composition, the unbound imager strands are partially double-stranded.

In some embodiments of the composition, imager strands are molecular beacons or comprise a hairpin secondary structure.

In some embodiments of the composition, the imager strands are molecular beacons or comprise a hairpin secondary structure that is self-quenching.

In some embodiments of the composition, wherein the imager strands comprise multiple (e.g., two such as, 3, 4, 5, 6, 7, 8, 9 10 or more) detectable labels.

In some embodiments of the composition, imager strands comprise a first detectable label at their 5′-end and a second detectable label at their 3′-end.

In some embodiments of the composition, the imager strand is from about 10 nucleotides to about 15 nucleotides in length, e.g., about 15 nucleotides in length.

In some embodiments of the composition, unbound barcode strands are partially double-stranded.

In some embodiments of the composition, unbound barcode strands comprise a hairpin secondary structure.

In some embodiments of the composition, the barcode strand is linked to the target-binding molecule via its 5′-end. In some other embodiments, the barcode strand is linked to the target-binding molecule via its 3′-end.

In some embodiments of the composition, the target-binding molecule is an antibody, a nucleic acid, a receptor, a ligand for a receptor, an antigen, or an enzyme.

In some embodiments of the composition, the target-binding molecule is an antibody or antibody fragment (e.g., antigen binding portion of an antibody) or a nucleic acid.

In some embodiments of the composition, the target-binding molecule is an antibody or antibody fragment (e.g., antigen binding portion of an antibody).

In some embodiments of the composition, the target-binding molecule is bound to its target.

In some embodiments, a composition can comprise a plurality of the same species or distinct species of target-molecules linked to a barcode strand, amplifier strands, adaptor strands and imager strands. In some embodiments, a composition can comprise at least 10, 50, 100, 500, 1000, 2000, 3000, 4000, 5000, 10⁴, 50000, 10⁵, 10⁵, 10⁶, 10⁷, 10⁸, 10⁹, 10¹⁰, 10¹¹ target-molecules linked to a barcode strand, amplifier strands, adaptor strands and imager strands. In some embodiments, a composition can comprise at least 10, 50, 100, 500, 1000, 2000, 3000, 4000, 5000, 10⁴, 50000, 10⁵, 10⁵, 10⁶, 10⁷, 10⁸, 10⁹, 10¹⁰, 10¹¹ target-molecules linked to a barcode strand, amplifier strands, adaptor strands and imager strands. In some embodiments, a composition can contain 1 to about 200 or more distinct species of target-molecules linked to a barcode strand, amplifier strands, adaptor strands and/or imager strands. For example, a composition can contain at least 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200 or more distinct species of target-molecules linked to a barcode strand, amplifier strands, adaptor strands and/or imager strands. In some embodiments, a composition can contain less than about 5 to about 200 distinct species of target-molecules linked to a barcode strand, amplifier strands, adaptor strands and imager strands. For example, a composition can contain less than 5, 6, 7, 8, 9, 10, 15, 20, 25, 30, 35, 40, 45, 50, 55, 60, 65, 70, 75, 80, 85, 90, 95, 100, 125, 150, 175 or 200 distinct species of target-molecules linked to a barcode strand, amplifier strands, adaptor strands and/or imager strands.

In some embodiments, a composition can comprise a plurality of the same species or distinct species of target-molecules linked to a barcode strand, amplifier strands, and imager strands. In some embodiments, a composition can comprise at least 10, 50, 100, 500, 1000, 2000, 3000, 4000, 5000, 104, 50000, 10⁵, 10⁵, 10⁶, 10⁷, 10⁸, 10⁹, 10¹⁰, 10¹¹ target-molecules linked to a barcode strand, amplifier strands, and imager strands. In some embodiments, a composition can comprise at least 10, 50, 100, 500, 1000, 2000, 3000, 4000, 5000, 104, 50000, 10⁵, 10⁵, 10⁶, 10⁷, 10⁸, 10⁹, 10¹⁰, 10¹¹ target-molecules linked to a barcode strand, amplifier strands, and imager strands. In some embodiments, a composition can contain 1 to about 200 or more distinct species of target-molecules linked to a barcode strand, amplifier strands, and/or imager strands. For example, a composition can contain at least 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200 or more distinct species of target-molecules linked to a barcode strand, amplifier strands, and/or imager strands. In some embodiments, a composition can contain less than about 5 to about 200 distinct species of target-molecules linked to a barcode strand, amplifier strands, and imager strands. For example, a composition can contain less than 5, 6, 7, 8, 9, 10, 15, 20, 25, 30, 35, 40, 45, 50, 55, 60, 65, 70, 75, 80, 85, 90, 95, 100, 125, 150, 175 or 200 distinct species of target-molecules linked to a barcode strand, amplifier strands, and/or imager strands.

### Kits

In another aspect, the present disclosure provides a kit comprising a composition, described herein.

In some embodiments, the kit comprises: (i) a first target-specific target binding molecule linked to a first barcode strand; (ii) a first amplifier strand comprising a barcode binding domain and a plurality of adapter binding domains, wherein the barcode binding domain comprises a nucleotide sequence that is substantially is substantially complementary to the first barcode strand; (iii) at least one first adaptor strand comprising an amplifier binding domain and at least one imager binding domain, wherein the amplifier domain comprises a nucleotide complementary to a nucleotide sequence of the adapter binding domain of the first amplifier strand; and (iv) at least one first imager strand comprising a first detectable label and a nucleotide sequence that is complementary to a nucleotide sequence of the imager binding domain of the first adaptor strand. In some embodiments, the kit further comprises (i) a second target-specific target binding molecule linked to a second barcode strand, wherein the second target-binding molecule binds to a target that is different from the first target-binding molecule; (ii) a second amplifier strand comprising a barcode binding domain and a plurality of adapter binding domains, wherein the barcode binding domain comprises a nucleotide sequence that is substantially is substantially complementary to the second barcode strand; (iii) at least one second adaptor strand comprising an amplifier binding domain and at least one imager binding domain, wherein the amplifier domain comprises a nucleotide complementary to a nucleotide sequence of the adapter binding domain of the second amplifier strand; and (iv) at least one second imager strand comprising a second detectable label and a nucleotide sequence that is complementary to a nucleotide sequence of the imager binding domain of the second adaptor strand, and wherein the first and second detectable labels are different.

In some embodiments, the kit comprises: (i) a first target-binding molecule linked to a first barcode strand; (ii) a first amplifier strands comprising a barcode binding domain, wherein the barcode binding domain comprises a nucleotide sequence that is substantially complementary to the first barcode strand; and (iii) a first imager strand comprising a first detectable label and a nucleotide sequence that is substantially complementary to a nucleotide sequence of the first imager binding domain. In some embodiments, the kit further comprises: (i) a second target-binding molecule linked to a second barcode strand, wherein the second target-binding molecule binds to a target that is different from the first target-binding molecule; (ii) a second amplifier strands comprising a barcode binding domain, wherein the barcode binding domain comprises a nucleotide sequence that is substantially complementary to the second barcode strand; and (iii) a second imager strand comprising a second detectable label and a nucleotide sequence that is substantially complementary to a nucleotide sequence of the second imager binding domain, and wherein the first and second detectable labels are different.

In some embodiments of the kit, a melting temperature of the imager strand binding/hybridizing with the adaptor strand is lower than a melting temperature of the amplifier strand binding/hybridizing with the barcode strand and the imager strand binding/hybridizing with the adaptor strand. For example, a melting temperature of imager strand binding/hybridizing with the adaptor strand is at least about 5° C. lower than a melting temperature of the amplifier strand binding/hybridizing with the barcode strand and the imager strand binding/hybridizing with the adaptor strand.

In some embodiments of the kit, the amplifier binding domain of the adaptor strand is at 5′ of the imager binding domain. In some other embodiments of the kit, the amplifier binding domain of the adaptor strand is at 3′ of the imager binding domain.

In some embodiments of the kit, the barcode binding domain of the amplifier strand is 5′ of the plurality of adapter binding domains. In some other embodiments of the kit, the barcode binding domain of the amplifier strand is 3′ of the plurality of adapter binding domains.

In some embodiments of the kit, a melting temperature of the imager strand binding/hybridizing with the adaptor strand is from about 35° C. to about 45° C.

In some embodiments of the kit, a melting temperature of the adaptor strand binding/hybridizing with the amplifier strand is at least 50° C. or higher.

In some embodiments of the kit, the amplifier strand comprises at least 5 (e.g., 5, 6, 7, 8, 9, 10, 11, 12, 13, 14, 15 or more) adaptor binding domains. For example, the amplifier strand comprises 10 adaptor binding domains.

In some embodiments of the kit, the imager strand is hybridized with the adaptor strand. In some embodiments of the kit, the adaptor strand is hybridized with the amplifier strand. In some embodiments of the kit, the imager strand is hybridized with the adaptor strand and the adaptor strand is hybridized with the amplifier strand.

In some embodiments of the kit, a melting temperature of imager strand binding/hybridizing with the amplifier strand is lower than the amplifier strand binding/hybridizing with the barcode strand. In some other embodiments of the kit, a melting temperature of imager strand binding/hybridizing with the amplifier strand is at least about 5° C. lower than the amplifier strand binding/hybridizing with the barcode strand.

In some embodiments of the kit, the barcode binding domain of the amplifier strand is 5′ of the plurality of imager binding domains. In some other embodiments, the barcode binding domain of the amplifier strand is 3′ of the plurality of imager binding domains.

In some embodiments of the kit, a melting temperature of the imager strand binding/hybridizing with the amplifier strand is from about 35° C. to about 45° C.

In some embodiments of the kit, the amplifier strand comprises at least 5 (e.g., 5, 6, 7, 8, 9, 10, 11, 12, 13, 14, 15 or more) imager binding domains.

In some embodiments of the kit, the imager strand is hybridized with the amplifier strand.

In some embodiments of the kit, a melting temperature of the amplifier strand binding/hybridizing with the barcode strand is at least 50° C. or higher.

In some embodiments of the kit, the detectable label is attached at the 5′-end of the imager strand it is attached to.

In some embodiments of the kit, detectable label is attached at the 3′-end of the imager strand it is attached to.

In some embodiments of the kit, the imager strand comprises, at its end the detectable label is attached to, at least two nucleotides that do not hybridized with the strand the imager strand is bound/hybridized to.

In some embodiments of the kit, the unbound imager strands are partially double-stranded.

In some embodiments of the kit, imager strands are molecular beacons or comprise a hairpin secondary structure.

In some embodiments of the kit, the imager strands are molecular beacons or comprise a hairpin secondary structure that is self-quenching.

In some embodiments of the kit, wherein the imager strands comprise multiple (e.g., two such as, 3, 4, 5, 6, 7, 8, 9 10 or more) detectable labels.

In some embodiments of the kit, imager strands comprise a first detectable label at their 5′-end and a second detectable label at their 3′-end.

In some embodiments of the kit, the imager strand is from about 10 nucleotides to about 15 nucleotides in length, e.g., about 15 nucleotides in length.

In some embodiments of the kit, unbound barcode strands are partially double-stranded.

In some embodiments of the kit, unbound barcode strands comprise a hairpin secondary structure.

In some embodiments of the kit, the barcode strand is linked to the target-binding molecule via its 5′-end. In some other embodiments, the barcode strand is linked to the target-binding molecule via its 3′-end.

In some embodiments of the kit, the target-binding molecule is an antibody, a nucleic acid, a receptor, a ligand for a receptor, an antigen, or an enzyme.

In some embodiments of the kit, the target-binding molecule is an antibody or antibody fragment (e.g., antigen binding portion of an antibody) or a nucleic acid.

In some embodiments of the kit, the target-binding molecule is an antibody or antibody fragment (e.g., antigen binding portion of an antibody).

In addition to the above mentioned components, any embodiments of the kits described herein can include informational material. The informational material can be descriptive, instructional, marketing or other material that relates to the methods described herein and/or the use of the aggregates for the methods described herein. For example, the informational material can describe methods for using the kits provided herein to perform an assay for capture and/or detection of a target analyte. The kit can also include an empty container and/or a delivery device, e.g., which can be used to deliver a test sample to a test container.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the formulation and/or its use in the methods described herein. Of course, the informational material can also be provided in any combination of formats.

In some embodiments, the kit can contain separate containers, dividers or compartments for each component and informational material. For example, each different component can be contained in a bottle, vial, or syringe, and the informational material can be contained in a plastic sleeve or packet. In other embodiments, the separate elements of the kit are contained within a single, undivided container.

### Applications

The compositions, kits and methods described herein can be used, inter alia, in any assay in which existing target detection technologies are used. Typically assays include detection assays including diagnostic assays, prognostic assays, patient monitoring assays, screening assays, bio-warfare assays, forensic analysis assays, prenatal genomic diagnostic assays and the like. The assay can be an in vitro assay or an in vivo assay. The present invention provides the advantage that many different targets can be analyzed at one time from a single sample using the methods of the invention, even where such targets are spatially not resolvable (and thus spatially indistinct) using prior art imaging methods. This allows, for example, for several diagnostic tests to be performed on one sample.

The compositions, kits and methods described herein can also be used to simply observe an area or region.

The compositions, kits and methods described herein can be applied to the analysis of samples obtained or derived from a patient so as to determine whether a diseased cell type is present in the sample and/or to stage the disease. For example, a blood sample can be assayed according to any of the methods described herein to determine the presence and/or quantity of markers of a cancerous cell type in the sample, thereby diagnosing or staging the cancer.

Alternatively, the compositions, kits and methods described herein can be used to diagnose pathogen infections, for example infections by intracellular bacteria and viruses, by determining the presence and/or quantity of markers of bacterium or virus, respectively, in the sample. Thus, the targets detected using the compositions, kits and methods described herein can be either patient markers (such as a cancer marker) or markers of infection with a foreign agent, such as bacterial or viral markers.

The quantitative imaging methods described herein can be used, for example, to quantify targets (e.g., target biomolecules) whose abundance is indicative of a biological state or disease condition (e.g., blood markers that are upregulated or down-regulated as a result of a disease state).

Further, the compositions, kits and methods described herein can be used to provide prognostic information that assists in determining a course of treatment for a patient. For example, the amount of a particular marker for a tumor can be accurately quantified from even a small sample from a patient. For certain diseases like breast cancer, overexpression of certain proteins, such as Her2-neu, indicate a more aggressive course of treatment will be needed.

The compositions, kits and methods described herein can also be used for determining the effect of a perturbation, including chemical compounds, mutations, temperature changes, growth hormones, growth factors, disease, or a change in culture conditions, on various targets, thereby identifying targets whose presence, absence or levels are indicative of a particular biological states. In some embodiments, the present invention is used to elucidate and discover components and pathways of disease states. For example, the comparison of quantities of targets present in a disease tissue with “normal” tissue allows the elucidation of important targets involved in the disease, thereby identifying targets for the discovery/screening of new drug candidates that can be used to treat disease.

The sample being analyzed can be a biological sample, such as blood, sputum, lymph, mucous, stool, urine and the like. The sample can be an environmental sample such as a water sample, an air sample, a food sample and the like. The assay can be carried out with one or more components of the binding reaction immobilized. Thus, the targets or the target-binding molecules linked with the barcode strand can be immobilized. The assay can be carried out with one or more components of the binding reaction non-immobilized. The assays can involve detection of a number of targets in a sample, essentially at the same time, in view of the multiplexing potential offered by the target-binding molecules linked with the barcode strand and fluorescently labeled imager strands described herein. As an example, an assay can be used to detect a particular cell type (e.g., based on a specific cell surface receptor) and a particular genetic mutation in that particular cell type. In this way, an end user can be able to determine how many cells of a particular type carry the mutation of interest, as an example.

Some exemplary aspects of the disclosure are described by one or more of following numbered Embodiments:

Embodiment 1: A method comprising:
(a) contacting a sample being tested for the presence of one or more targets with one or more target-specific target-binding molecules, wherein each target-binding molecule is linked to a barcode strand, and wherein target-binding molecules of different specificity are linked to different barcode strands,
(b) optionally removing unbound target-binding molecules,
(c) contacting the sample with amplifier strands comprising a barcode binding domain and a plurality of adapter binding domains, wherein the barcode binding domain comprises a nucleotide sequence that is substantially complementary to a barcode strand,
(d) optionally removing unbound amplifier strands,
(e) contacting the sample with a first set of adaptor strands bound/hybridized with imager strands, wherein the adaptor strands comprise an amplifier binding domain and at least one imager binding domain, wherein the amplifier domain comprises a nucleotide complementary to a nucleotide sequence of an adapter binding domain, and wherein the imager strands comprising a detectable label and a nucleotide sequence that is complementary to a nucleotide sequence of an imager binding domain,
(f) optionally removing unbound adapter strands and imager strands,
(g) imaging the sample to detect location of bound imager strands,
(h) extinguishing a detectable signal from the detectable labels of the bound imager strands, and
(i) optionally, repeating steps (e)-(h) at least once with a second set of adaptor strands bound/hybridized with imager strands and having a unique nucleotide sequence relative to the nucleotide sequences of the adaptor strands in the first set.

Embodiment 2: A method comprising:
(a) contacting a sample being tested for the presence of one or more targets with one or more target-specific target-binding molecules, wherein each target-binding molecule is linked to a barcode strand, and wherein target-binding molecules of different specificity are linked to different barcode strands,
(b) optionally removing unbound target-binding molecules,
(c) contacting the sample with amplifier strands comprising a barcode binding domain and a plurality of adapter binding domains, wherein the barcode binding domain comprises a nucleotide sequence that is substantially complementary to a barcode strand,
(d) optionally removing unbound amplifier strands,
(e) contacting the sample with a first set of adaptor strands comprising an amplifier binding domain and at least one imager binding domain, wherein the amplifier domain comprises a nucleotide complementary to a nucleotide sequence of an adapter binding domain,
(f) optionally removing unbound adapter strands,
(g) contacting the sample with imager strands comprising a detectable label and a nucleotide sequence that is complementary to a nucleotide sequence of an imager binding domain,
(h) optionally removing unbound imager strands,
(i) imaging the sample to detect location of bound imager strands,
(j) extinguishing a detectable signal from the detectable labels of the bound imager strands, and
(k) optionally, repeating steps (e)-(j) at least once with a second set of adaptor strands having a unique nucleotide sequence relative to the nucleotide sequences of the adaptor strands in the first set.

Embodiment 3: The method of any one of the preceding Embodiments, wherein said step of extinguishing the signal from the bound imager strands comprises removing the adaptor strands from the amplifier strands they are hybridized to, or removing the detectable label from the bound imager strands, or modifying the detectable label.

Embodiment 4: The method of any one of the preceding Embodiments, wherein said step of extinguishing the signal from the bound imager strands comprises removing the bound adaptor strands from the amplifier strands they are hybridized to by altering temperature, by altering buffer conditions and/or addition of a complementary nucleic acid strand.

Embodiment 5: The method of any one of the preceding Embodiments, wherein the adaptor strands are removed from the amplifier strand by addition of a denaturant, increasing temperature, addition of a complementary nucleic acid strand, and/or decreasing salt concentration.

Embodiment 6: The method of any one of the preceding Embodiments, wherein the adaptor strands are removed from the amplifier strand by addition of a denaturant selected from the group consisting of formamide, urea, and DMSO.

Embodiment 7: The method of any one of Embodiments 1-5, wherein the adaptor strands are removed from the amplifier strand by addition of complementary nucleic acid strands, wherein the nucleic acid strands comprise a nucleotide sequence substantially complementary to amplifier binding domain of the adaptor strand or the adaptor binding domain of the amplifier strand.

Embodiment 8: The method of any one of Embodiments 1-5, wherein said step of extinguishing the signal from the bound imager strands comprises removing or modifying the detectable label without removing the adaptor strands.

Embodiment 9: The method of any one of Embodiments 1-5 or 8, wherein said step of extinguishing the signal from the bound imager strands comprises photobleaching.

Embodiment 10: The method of any one Embodiments 1-5 or 8, wherein said step of extinguishing the signal from the bound imager strands comprises cleaving the detectable label from the imager strand.

Embodiment 11: The method of Embodiment 10, wherein said cleaving the detectable label from the imager strand comprises enzymatic cleavage, chemical cleavage or photo cleavage.

Embodiment 12: The method of any one of the preceding Embodiments, wherein at least one adapter strand in the first set of the adaptor strands binds/hybridizes with a first amplifier strand and at least one other adapter strand in the first set of the adaptor strands binds/hybridizes with a second amplifier strand, and wherein the first and second amplifier strand are bound/hybridized to barcode strands linked to target-binding molecules of different specificity (i.e., target-binding molecules bind to different targets).

Embodiment 13: The method of any one of the preceding Embodiments, wherein at least one adapter strand in the second set of the adaptor strands binds/hybridizes with a first amplifier strand and at least one other adapter strand in the second set of the adaptor strands binds/hybridizes with second amplifier strand, and wherein the first and second amplifier strand are bound/hybridized to barcode strands linked to target-binding molecules of different specificity (i.e., target-binding molecules bind to different targets).

Embodiment 14: The method of any one of the preceding Embodiments, wherein at least one imager strand binds to an adaptor strand binds/hybridizes with a first amplifier strand and at least one other adaptor strand binds/hybridizes with a second amplifier strand, and wherein the first and second amplifier strand are bound/hybridized to barcode strands linked to target-binding molecules of different specificity (i.e., target-binding molecules bind to different targets), and wherein the detectable labels of the imager strands are different.

Embodiment 15: The method of any one of the preceding Embodiments, wherein a melting temperature (Tₘ) of imager strand binding/hybridizing with the adaptor strand is lower than a melting temperature of the amplifier strand binding/hybridizing with the barcode strand and the imager strand binding/hybridizing with the adaptor strand.

Embodiment 16: The method of any one of the preceding Embodiments, wherein a melting temperature (Tₘ) of imager strand binding/hybridizing with the adaptor strand is at least about 5° C. lower than a melting temperature of the amplifier strand binding/hybridizing with the barcode strand and the imager strand binding/hybridizing with the adaptor strand.

Embodiment 17: The method of any one of the preceding Embodiments, wherein the amplifier binding domain of the adaptor strand is at 5′ of the imager binding domain.

Embodiment 18: The method of any one of Embodiments 1-16, wherein the amplifier binding domain of the adaptor strand is at 3′ of the imager binding domain.

Embodiment 19: The method of any one of the preceding Embodiments, wherein the barcode binding domain of the amplifier strand is 5′ of the plurality of adapter binding domains.

Embodiment 20: The method of any one of Embodiments 1-16, wherein the barcode binding domain of the amplifier strand is 3′ of the plurality of adapter binding domains.

Embodiment 21: A method comprising:
(a) contacting a sample being tested for the presence of one or more targets with one or more target-specific target-binding molecules, wherein each target-binding molecule is linked to a barcode strand, and wherein target-binding molecules of different specificity are linked to different barcode strands,
(b) optionally removing unbound target-binding molecules,
(c) contacting the sample with amplifier strands comprising a barcode binding domain, wherein the barcode binding domain comprises a nucleotide sequence that is substantially complementary to a barcode strand,
(d) optionally removing unbound amplifier strands,
(e) contacting the sample with a first set of imager strands comprising a detectable label and a nucleotide sequence that is substantially complementary to a nucleotide sequence of an imager binding domain,
(f) optionally removing unbound imager strands,
(g) imaging the sample to detect location of bound labeled imager strands,
(h) extinguishing a detectable signal from the bound labeled imager strands, and
(i) optionally, repeating steps (e)-(h), each time with second set of imager strands having a unique nucleotide sequence relative to the imager strands of the first set.

Embodiment 22: The method of Embodiment 21, wherein said step of extinguishing the signal from the bound imager strands comprises removing the imager strands from the amplifier strands they are hybridized to, or removing the detectable label from the bound imager strands, or modifying the detectable label.

Embodiment 23: The method of Embodiment 21 or 22, wherein said step of extinguishing the signal from the bound imager strands comprises removing the bound imager strands from the amplifier strands they are hybridized to by altering temperature, by altering buffer conditions and/or addition of a complementary nucleic acid strand.

Embodiment 24: The method of any one of Embodiments 21-23, wherein the imager strands are removed from the amplifier strand by addition of a denaturant, increasing temperature, addition of a complementary nucleic acid strand, and/or decreasing salt concentration.

Embodiment 25: The method of any one of Embodiments 21-24, wherein the imager strands are removed from the amplifier strand by addition of a denaturant selected from the group consisting of formamide, urea, and DMSO.

Embodiment 26: The method of any one of Embodiments 21-23, wherein the imager strands are removed from the amplifier strand by addition of complementary nucleic acid strands, wherein the nucleic acid strands comprise a nucleotide sequence substantially complementary to amplifier binding domain of the imager strand or the imager binding domain of the amplifier strand

Embodiment 27: The method of any one of Embodiments 21-23, wherein said step of extinguishing the signal from the bound imager strands comprises removing or modifying the detectable label without removing the imager strands.

Embodiment 28: The method of any one of Embodiments 21-23 or 27, wherein said step of extinguishing the signal from the bound imager strands comprises photobleaching.

Embodiment 29: The method of any one of Embodiments 21-23 or 27, wherein said step of extinguishing the signal from the bound imager strands comprises cleaving the detectable label from the imager strand.

Embodiment 30: The method of Embodiment 29, wherein said cleaving the detectable label from the imager strand comprises enzymatic cleavage, chemical cleavage or photo cleavage.

Embodiment 31: The method of any one of Embodiments 21-30, wherein at least one imager strand in the first set of the imager strands binds/hybridizes with a first amplifier strand and at least one other imager strand in the first set of the imager strands binds/hybridizes with second amplifier strand, and wherein the first and second amplifier strand are bound/hybridized to barcode strands linked to target-binding molecules of different specificity (i.e., target-binding molecules bind to different targets), and wherein the detectable labels are different.

Embodiment 32: The method of any one of Embodiments 21-31, wherein at least one imager strand in the second set of the imager strands binds/hybridizes with a first amplifier strand and at least one other imager strand in the second set of the adaptor strands binds/hybridizes with a second amplifier strand, and wherein the first and second amplifier strand are bound/hybridized to barcode strands linked to target-binding molecules of different specificity (i.e., target-binding molecules bind to different targets) and wherein the detectable labels are different.

Embodiment 33: The method of any one of Embodiments 21-32, wherein a melting temperature of imager strand binding/hybridizing with the amplifier strand is lower than the amplifier strand binding/hybridizing with the barcode strand.

Embodiment 34: The method of any one of Embodiments 21-33, wherein a melting temperature of imager strand binding/hybridizing with the amplifier strand is at least about 5° C. lower than the amplifier strand binding/hybridizing with the barcode strand.

Embodiment 35: The method of any one of Embodiments 21-34, wherein the barcode binding domain of the amplifier strand is 5′ of the plurality of imager binding domains.

Embodiment 36: The method of any one of Embodiments 21-34, wherein the barcode binding domain of the amplifier strand is 3′ of the plurality of imager binding domains.

Embodiment 37: The method of any one of the preceding Embodiments, wherein the detectable label is attached at the 5′-end of the imager strand it is attached to.

Embodiment 38: The method of any one of Embodiments 1-36, wherein the detectable label is attached at the 3′-end of the imager strand it is attached to.

Embodiment 39: The method of any one of the preceding Embodiments, wherein the imager strand comprises, at its end the detectable label is attached to, at least two nucleotides that do not hybridized with the strand the imager strand is bound/hybridized to.

Embodiment 40: The method of any one of the preceding Embodiments, wherein the unbound imager strands are partially double-stranded.

Embodiment 41: The method of any one of the preceding Embodiments, wherein the imager strands are molecular beacons or comprise a hairpin secondary structure.

Embodiment 42: The method of any one of the preceding Embodiments, wherein the imager strands are molecular beacons or comprise a hairpin secondary structure that is self-quenching.

Embodiment 43: The method of any one of the preceding Embodiments, wherein the imager strands comprise multiple detectable labels.

Embodiment 44: The method of any one of the preceding Embodiments, wherein the imager strands comprise a first detectable label at their 5′-end and a second detectable label at their 3′-end.

Embodiment 45: The method of any one of Embodiments, wherein the imager strand is from about 10 nucleotides to about 15 nucleotides in length, e.g., about 15 nucleotides in length.

Embodiment 46: The method of any one of the preceding Embodiments, wherein unbound barcode strands are partially double-stranded.

Embodiment 47: The method of any one of the preceding Embodiments, wherein unbound barcode strands comprise a hairpin secondary structure.

Embodiment 48: The method of any one of the preceding Embodiments, wherein the barcode strand is linked to the target-binding molecule via its 5′-end.

Embodiment 49: The method of any one of Embodiments 1-47, wherein the barcode strand is linked to the target-binding molecule via its 3′-end.

Embodiment 50: The method of any one of the preceding Embodiments, wherein the target-binding molecule is an antibody, a nucleic acid, a receptor, a ligand for a receptor, an antigen, or an enzyme.

Embodiment 51: The method of any one of the preceding Embodiments, wherein the sample is contacted with more than one target-binding molecule in step (a).

Embodiment 52: The method of any one of the preceding Embodiments, wherein the target-binding molecule is an antibody or antibody fragment (e.g., antigen binding portion of an antibody) or a nucleic acid.

Embodiment 53: The method of any one of the preceding Embodiments, wherein the target-binding molecule is an antibody or antibody fragment (e.g., antigen binding portion of an antibody).

Embodiment 54: The method of any one of the preceding Embodiments, wherein the sample is imaged using confocal or epi-fluorescence microscopy.

Embodiment 55: A composition comprising: (i) a first target-specific target binding molecule linked to a first barcode strand; (ii) a first amplifier strand comprising a barcode binding domain and a plurality of adapter binding domains, wherein the barcode binding domain comprises a nucleotide sequence that is substantially is substantially complementary to the first barcode strand; (iii) at least one first adaptor strand comprising an amplifier binding domain and at least one imager binding domain, wherein the amplifier domain comprises a nucleotide complementary to a nucleotide sequence of the adapter binding domain of the first amplifier strand; and (iv) at least one first imager strand comprising a first detectable label and a nucleotide sequence that is complementary to a nucleotide sequence of the imager binding domain of the first adaptor strand.

Embodiment 56: The composition of Embodiment 55, wherein the composition further comprises (i) a second target-specific target binding molecule linked to a second barcode strand, wherein the second target-binding molecule binds to a target that is different from the first target-binding molecule; (ii) a second amplifier strand comprising a barcode binding domain and a plurality of adapter binding domains, wherein the barcode binding domain comprises a nucleotide sequence that is substantially is substantially complementary to the second barcode strand; (iii) at least one second adaptor strand comprising an amplifier binding domain and at least one imager binding domain, wherein the amplifier domain comprises a nucleotide complementary to a nucleotide sequence of the adapter binding domain of the second amplifier strand; and (iv) at least one second imager strand comprising a second detectable label and a nucleotide sequence that is complementary to a nucleotide sequence of the imager binding domain of the second adaptor strand, and wherein the first and second detectable labels are different.

Embodiment 57: The composition of any one of the preceding Embodiments, wherein a melting temperature of the imager strand binding/hybridizing with the adaptor strand is lower than a melting temperature of the amplifier strand binding/hybridizing with the barcode strand and the imager strand binding/hybridizing with the adaptor strand.

Embodiment 58: The composition of any one of the preceding Embodiments, wherein a melting temperature of imager strand binding/hybridizing with the adaptor strand is at least about 5° C. lower than a melting temperature of the amplifier strand binding/hybridizing with the barcode strand and the imager strand binding/hybridizing with the adaptor strand.

Embodiment 59: The composition of any one of the preceding Embodiments, wherein the amplifier binding domain of the adaptor strand is at 5′ of the imager binding domain.

Embodiment 60: The composition of any one of Embodiments 55-59, wherein the amplifier binding domain of the adaptor strand is at 3′ of the imager binding domain.

Embodiment 61: The composition of any one of the preceding Embodiments, wherein the barcode binding domain of the amplifier strand is 5′ of the plurality of adapter binding domains.

Embodiment 62: The method of any one of Embodiments 55-60, wherein the barcode binding domain of the amplifier strand is 3′ of the plurality of adapter binding domains.

Embodiment 63: The composition of any one of the preceding Embodiments, wherein a melting temperature of the imager strand binding/hybridizing with the adaptor strand is from about 35° C. to about 45° C.

Embodiment 64: The composition of any one of the preceding Embodiments, wherein a melting temperature of the adaptor strand binding/hybridizing with the amplifier strand is at least 50° C. or higher.

Embodiment 65: The composition of any one of the preceding Embodiments, wherein the amplifier strand comprises at least 5 (e.g., 5, 6, 7, 8, 9, 10, 11, 12, 13, 14, 15 or more) adaptor binding domains.

Embodiment 66: The composition of any one of the preceding Embodiments, wherein the imager strand is hybridized with the adaptor strand.

Embodiment 67: The composition of any one of the preceding Embodiments, wherein the adaptor strand is hybridized with the amplifier strand.

Embodiment 68: A composition comprising: (i) a first target-binding molecule linked to a first barcode strand; (ii) a first amplifier strands comprising a barcode binding domain, wherein the barcode binding domain comprises a nucleotide sequence that is substantially complementary to the first barcode strand; and (iii) a first imager strand comprising a first detectable label and a nucleotide sequence that is substantially complementary to a nucleotide sequence of the first imager binding domain.

Embodiment 69: The composition of Embodiment 68, wherein the composition further comprises: (i) a second target-binding molecule linked to a second barcode strand, wherein the second target-binding molecule binds to a target that is different from the first target-binding molecule; (ii) a second amplifier strands comprising a barcode binding domain, wherein the barcode binding domain comprises a nucleotide sequence that is substantially complementary to the second barcode strand; and (iii) a second imager strand comprising a second detectable label and a nucleotide sequence that is substantially complementary to a nucleotide sequence of the second imager binding domain, and wherein the first and second detectable labels are different.

Embodiment 70: The composition of Embodiment 68 or 69, wherein a melting temperature of imager strand binding/hybridizing with the amplifier strand is lower than the amplifier strand binding/hybridizing with the barcode strand.

Embodiment 71: The composition of any one of Embodiments 68-70, wherein a melting temperature of imager strand binding/hybridizing with the amplifier strand is at least about 5° C. lower than the amplifier strand binding/hybridizing with the barcode strand.

Embodiment 72: The composition of any one of Embodiments 68-71, wherein the barcode binding domain of the amplifier strand is 5′ of the plurality of imager binding domains.

Embodiment 73: The composition of any one of Embodiments 68-72, wherein the barcode binding domain of the amplifier strand is 3′ of the plurality of imager binding domains.

Embodiment 74: The composition of any one of Embodiments 68-73, wherein a melting temperature of the imager strand binding/hybridizing with the amplifier strand is from about 35° C. to about 45° C.

Embodiment 75: The composition of any one of Embodiments 68-74, wherein the amplifier strand comprises at least 5 (e.g., 5, 6, 7, 8, 9, 10, 11, 12, 13, 14, 15 or more) imager binding domains.

Embodiment 76: The composition of any one of Embodiments 68-75, wherein the imager strand is hybridized with the amplifier strand.

Embodiment 77: The composition of any one of the preceding Embodiments, wherein a melting temperature of the amplifier strand binding/hybridizing with the barcode strand is at least 50° C. or higher.

Embodiment 78: The composition of any one of the preceding Embodiments, wherein the detectable label is attached at the 5′-end of the imager strand it is attached to.

Embodiment 79: The composition of any one of Embodiments 55-78, wherein the detectable label is attached at the 3′-end of the imager strand it is attached to.

Embodiment 80: The composition of any one of the preceding Embodiments, wherein the imager strand comprises, at its end the detectable label is attached to, at least two nucleotides that do not hybridized with the strand the imager strand is bound/hybridized to.

Embodiment 81: The composition of any one of the preceding Embodiments, wherein the unbound imager strands are partially double-stranded.

Embodiment 82: The composition of any one of the preceding Embodiments, wherein the imager strands are molecular beacons or comprise a hairpin secondary structure.

Embodiment 83: The composition of any one of the preceding Embodiments, wherein the imager strands are molecular beacons or comprise a hairpin secondary structure that is self-quenching.

Embodiment 84: The composition of any one of the preceding Embodiments, wherein the imager strands comprise multiple (e.g., two or more) detectable labels.

Embodiment 85: The composition of any one of the preceding Embodiments, wherein the imager strands comprise a first detectable label at their 5′-end and a second detectable label at their 3′-end.

Embodiment 86: The composition of any one of Embodiments, wherein the imager strand is from about 10 nucleotides to about 15 nucleotides in length, e.g., about 15 nucleotides in length.

Embodiment 87: The composition of any one of the preceding Embodiments, wherein unbound barcode strands are partially double-stranded.

Embodiment 88: The composition of any one of the preceding Embodiments, wherein unbound barcode strands comprise a hairpin secondary structure.

Embodiment 89: The composition of any one of the preceding Embodiments, wherein the barcode strand is linked to the target-binding molecule via its 5′-end.

Embodiment 90: The composition of any one of Embodiments 55-89, wherein the barcode strand is linked to the target-binding molecule via its 3′-end.

Embodiment 91: The composition of any one of the preceding Embodiments, wherein the target-binding molecule is an antibody, a nucleic acid, a receptor, a ligand for a receptor, an antigen, or an enzyme.

Embodiment 92: The composition of any one of the preceding Embodiments, wherein the target-binding molecule is an antibody or antibody fragment (e.g., antigen binding portion of an antibody) or a nucleic acid.

Embodiment 93: The composition of any one of the preceding Embodiments, wherein the target-binding molecule is an antibody or antibody fragment (e.g., antigen binding portion of an antibody).

Embodiment 94: The composition of any one of the preceding Embodiments, wherein the target-binding molecule is bound to its target.

Embodiment 95: A kit comprising: (i) a first target-specific target binding molecule linked to a first barcode strand; (ii) a first amplifier strand comprising a barcode binding domain and a plurality of adapter binding domains, wherein the barcode binding domain comprises a nucleotide sequence that is substantially is substantially complementary to the first barcode strand; (iii) at least one first adaptor strand comprising an amplifier binding domain and at least one imager binding domain, wherein the amplifier domain comprises a nucleotide complementary to a nucleotide sequence of the adapter binding domain of the first amplifier strand; and (iv) at least one first imager strand comprising a first detectable label and a nucleotide sequence that is complementary to a nucleotide sequence of the imager binding domain of the first adaptor strand.

Embodiment 96: The kit of Embodiment 95, wherein the kit further comprises (i) a second target-specific target binding molecule linked to a second barcode strand, wherein the second target-binding molecule binds to a target that is different from the first target-binding molecule; (ii) a second amplifier strand comprising a barcode binding domain and a plurality of adapter binding domains, wherein the barcode binding domain comprises a nucleotide sequence that is substantially is substantially complementary to the second barcode strand; (iii) at least one second adaptor strand comprising an amplifier binding domain and at least one imager binding domain, wherein the amplifier domain comprises a nucleotide complementary to a nucleotide sequence of the adapter binding domain of the second amplifier strand; and (iv) at least one second imager strand comprising a second detectable label and a nucleotide sequence that is complementary to a nucleotide sequence of the imager binding domain of the second adaptor strand, and wherein the first and second detectable labels are different.

Embodiment 97: The kit of any one of the preceding Embodiments, wherein a melting temperature of the imager strand binding/hybridizing with the adaptor strand is lower than a melting temperature of the amplifier strand binding/hybridizing with the barcode strand and the imager strand binding/hybridizing with the adaptor strand.

Embodiment 98: The kit of any one of the preceding Embodiments, wherein a melting temperature of imager strand binding/hybridizing with the adaptor strand is at least about 5° C. lower than a melting temperature of the amplifier strand binding/hybridizing with the barcode strand and the imager strand binding/hybridizing with the adaptor strand.

Embodiment 99: The kit of any one of the preceding Embodiments, wherein the amplifier binding domain of the adaptor strand is at 5′ of the imager binding domain.

Embodiment 100: The kit of any one of Embodiments 95-98, wherein the amplifier binding domain of the adaptor strand is at 3′ of the imager binding domain.

Embodiment 101: The kit of any one of the preceding Embodiments, wherein the barcode binding domain of the amplifier strand is 5′ of the plurality of adapter binding domains.

Embodiment 102: The method of any one of Embodiments 95-100, wherein the barcode binding domain of the amplifier strand is 3′ of the plurality of adapter binding domains.

Embodiment 103: The kit of any one of the preceding Embodiments, wherein a melting temperature of the imager strand binding/hybridizing with the adaptor strand is from about 35° C. to about 45° C.

Embodiment 104: The kit of any one of the preceding Embodiments, wherein a melting temperature of the adaptor strand binding/hybridizing with the amplifier strand is at least 50° C. or higher.

Embodiment 105: The kit of any one of the preceding Embodiments, wherein the amplifier strand comprises at least 5 (e.g., 5, 6, 7, 8, 9, 10, 11, 12, 13, 14, 15 or more) adaptor binding domains.

Embodiment 106: The kit of any one of the preceding Embodiments, wherein the imager strand is hybridized with the adaptor strand.

Embodiment 107: The kit of any one of the preceding Embodiments, wherein the adaptor strand is hybridized with the amplifier strand.

Embodiment 108: A kit comprising: (i) a first target-binding molecule linked to a first barcode strand; (ii) a first amplifier strands comprising a barcode binding domain, wherein the barcode binding domain comprises a nucleotide sequence that is substantially complementary to the first barcode strand; and (iii) a first imager strand comprising a first detectable label and a nucleotide sequence that is substantially complementary to a nucleotide sequence of the first imager binding domain.

Embodiment 109: The kit of Embodiment 108, wherein the kit further comprises: (i) a second target-binding molecule linked to a second barcode strand, wherein the second target-binding molecule binds to a target that is different from the first target-binding molecule; (ii) a second amplifier strands comprising a barcode binding domain, wherein the barcode binding domain comprises a nucleotide sequence that is substantially complementary to the second barcode strand; and (iii) a second imager strand comprising a second detectable label and a nucleotide sequence that is substantially complementary to a nucleotide sequence of the second imager binding domain, and wherein the first and second detectable labels are different.

Embodiment 110: The kit of Embodiment 108 or 109, wherein a melting temperature of imager strand binding/hybridizing with the amplifier strand is lower than the amplifier strand binding/hybridizing with the barcode strand.

Embodiment 111: The kit of any one of Embodiments 108-110, wherein a melting temperature of imager strand binding/hybridizing with the amplifier strand is at least about 5° C. lower than the amplifier strand binding/hybridizing with the barcode strand.

Embodiment 112: The kit of any one of Embodiments 108-111, wherein the barcode binding domain of the amplifier strand is 5′ of the plurality of imager binding domains.

Embodiment 113: The kit of any one of Embodiments 108-112, wherein the barcode binding domain of the amplifier strand is 3′ of the plurality of imager binding domains.

Embodiment 114: The kit of any one of Embodiments 108-113, wherein a melting temperature of the imager strand binding/hybridizing with the amplifier strand is from about 35° C. to about 45° C.

Embodiment 115: The kit of any one of Embodiments 108-114, wherein the amplifier strand comprises at least 5 (e.g., 5, 6, 7, 8, 9, 10, 11, 12, 13, 14, 15 or more) imager binding domains.

Embodiment 116: The kit of any one of Embodiments 108-75, wherein the imager strand is hybridized with the amplifier strand.

Embodiment 117: The kit of any one of the preceding Embodiments, wherein a melting temperature of the amplifier strand binding/hybridizing with the barcode strand is at least 50° C. or higher.

Embodiment 118: The kit of any one of the preceding Embodiments, wherein the detectable label is attached at the 5′-end of the imager strand it is attached to.

Embodiment 119: The kit of any one of Embodiments 95-118, wherein the detectable label is attached at the 3′-end of the imager strand it is attached to.

Embodiment 120: The kit of any one of the preceding Embodiments, wherein the imager strand comprises, at its end the detectable label is attached to, at least two nucleotides that do not hybridized with the strand the imager strand is bound/hybridized to.

Embodiment 121: The kit of any one of the preceding Embodiments, wherein the unbound imager strands are partially double-stranded.

Embodiment 122: The kit of any one of the preceding Embodiments, wherein the imager strands are molecular beacons or comprise a hairpin secondary structure.

Embodiment 123: The kit of any one of the preceding Embodiments, wherein the imager strands are molecular beacons or comprise a hairpin secondary structure that is self-quenching.

Embodiment 124: The kit of any one of the preceding Embodiments, wherein the imager strands comprise multiple (e.g., two or more) detectable labels.

Embodiment 125: The kit of any one of the preceding Embodiments, wherein the imager strands comprise a first detectable label at their 5′-end and a second detectable label at their 3′-end.

Embodiment 126: The kit of any one of Embodiments, wherein the imager strand is from about 10 nucleotides to about 15 nucleotides in length, e.g., about 15 nucleotides in length.

Embodiment 127: The kit of any one of the preceding Embodiments, wherein unbound barcode strands are partially double-stranded.

Embodiment 128: The kit of any one of the preceding Embodiments, wherein unbound barcode strands comprise a hairpin secondary structure.

Embodiment 129: The kit of any one of the preceding Embodiments, wherein the barcode strand is linked to the target-binding molecule via its 5′-end.

Embodiment 130: The kit of any one of Embodiments 95-128, wherein the barcode strand is linked to the target-binding molecule via its 3′-end.

Embodiment 131: The kit of any one of the preceding Embodiments, wherein the target-binding molecule is an antibody, a nucleic acid, a receptor, a ligand for a receptor, an antigen, or an enzyme.

Embodiment 132: The kit of any one of the preceding Embodiments, wherein the target-binding molecule is an antibody or antibody fragment (e.g., antigen binding portion of an antibody) or a nucleic acid.

Embodiment 133: The kit of any one of the preceding Embodiments, wherein the target-binding molecule is an antibody or antibody fragment (e.g., antigen binding portion of an antibody).

Embodiment 134: The kit of any one of the preceding Embodiments, wherein the target-binding molecule is bound to its target.

Embodiment 135: The kit of any one of Embodiments 95-134, wherein the kit further comprises a blocker strand.

Embodiment 136: The composition of any one of Embodiments 55-94, wherein the composition further comprises a blocker strand,

Embodiment 137: The method of any one of claims 1-54, wherein said step of contacting the sample with the imager strands is in presence of blocker strands.

Embodiment 138: The method of Embodiment 137, wherein a concentration of the blocker strands is lower than a concentration of the imager strands.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials can be used in the practice or testing of the invention, the methods, devices, and materials in this regard are described herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not. In other words, except where the context requires otherwise, the term “comprise” and variations of the term, such as “comprising”, “comprises” and “comprised”, are not intended to exclude further additives, components, integers or steps.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “binding” or “bound” generally refers to a reversible binding of one molecule to molecule via, e.g., van der Waals force, hydrophobic force, hydrogen bonding, and/or electrostatic force. The binding interaction between two molecules can be described by a dissociation constant (K_{d}) or association constant (K).

Specific elements of any of the disclosed embodiments can be combined or substituted for elements in other embodiments. Furthermore, while advantages associated with certain embodiments of the disclosure have been described in the context of these embodiments, other embodiments can also exhibit such advantages, and not all embodiments need necessarily exhibit such advantages to fall within the scope of the disclosure.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1: Simple DNA-Based Multiplexable Amplification (SIMPL) Amplification Affords Comparable Amplification to Secondary Antibody

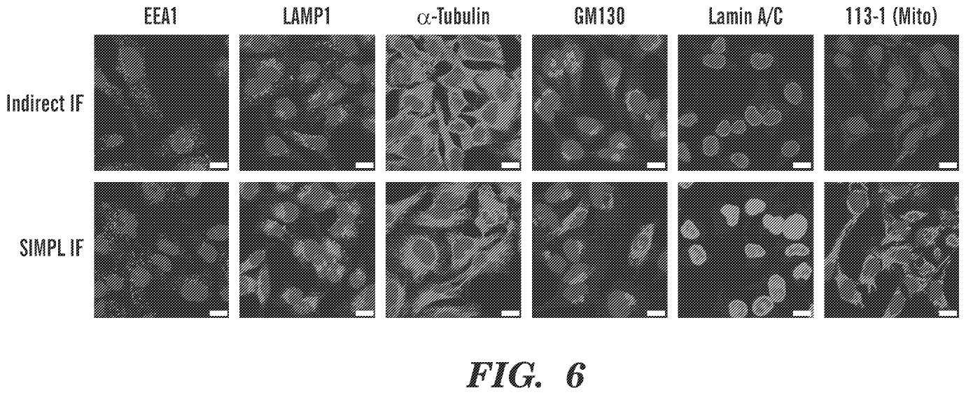

Formaldehyde-fixed Hela cells were imaged with six different antibodies targeting α-Tubulin, EEA1, LAMP1, Lamin A/C, GM130, and mitochondria (113-1), using classical indirect immunofluorescence (top row) and SIMPL immunofluorescence (bottom row). In both cases, fixed and permeabilized cells were incubated with primary antibody overnight. For indirect immunofluorescence, secondary antibody was added at 4 μg/mL for 30 minutes, in accordance with standard protocols. For SIMPL, DNA amplifiers in 2× SSC+10% dextran sulfate+30% formamide were added for one hour at 37° C., and imagers were added in 2× SSC +10% formamide for ten minutes at room temperature. Results are shown in FIG. 6. The same microscope and display settings for each target. Signal was equivalent or brighter in images created with SIMPL versus those created with indirect immunofluorescence. Scale bars are 10 μm.

### Example 2: SIMPL Multiplexing Proof-Of-Concept: Image with 6 Protein Targets

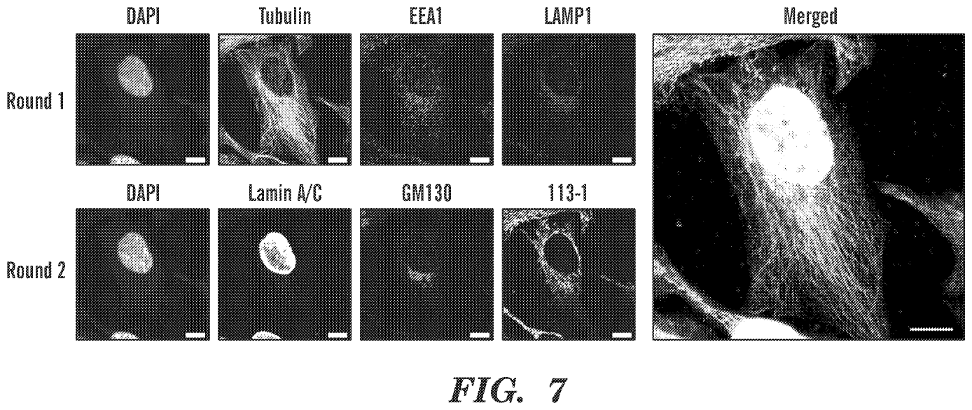

Formaldehyde-fixed Hela cells were incubated overnight at 4° C. with six DNA-barcoded antibodies targeting α-Tubulin, EEA1, LAMP1, Lamin A/C, GM130, and mitochondria (113-1). All DNA amplifiers in 2× SSC+10% dextran sulfate+30% formamide were added for one hour at 37° C. Then, the first three imagers were added in 2× SSC+10% formamide for ten minutes at room temperature. The sample was then imaged on a Zeiss LSM980 with a 63× oil objective in Airyscan mode (Round 1). After imaging, the imagers were stripped with five 2-minute washes with 2× SSC+45% formamide. The second set of imagers were added, the sample was returned to the microscope stage in the same location and imaged again (Round 2). Results are shown in FIG. 7. Individual images as well as a merged six-color image are displayed.

### Example 3: SIMPL Multiplexing Proof-Of-Concept 2: Image with 24 Protein Targets

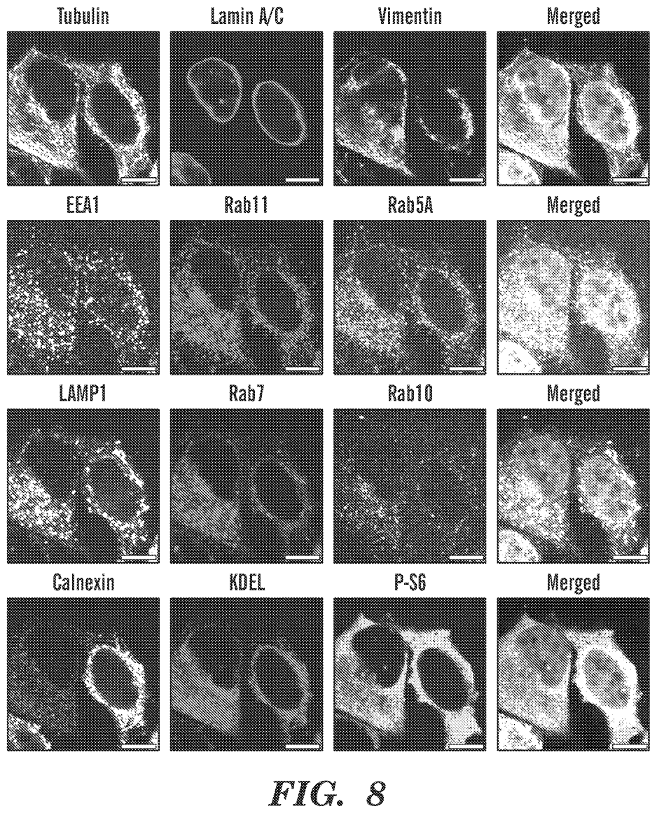

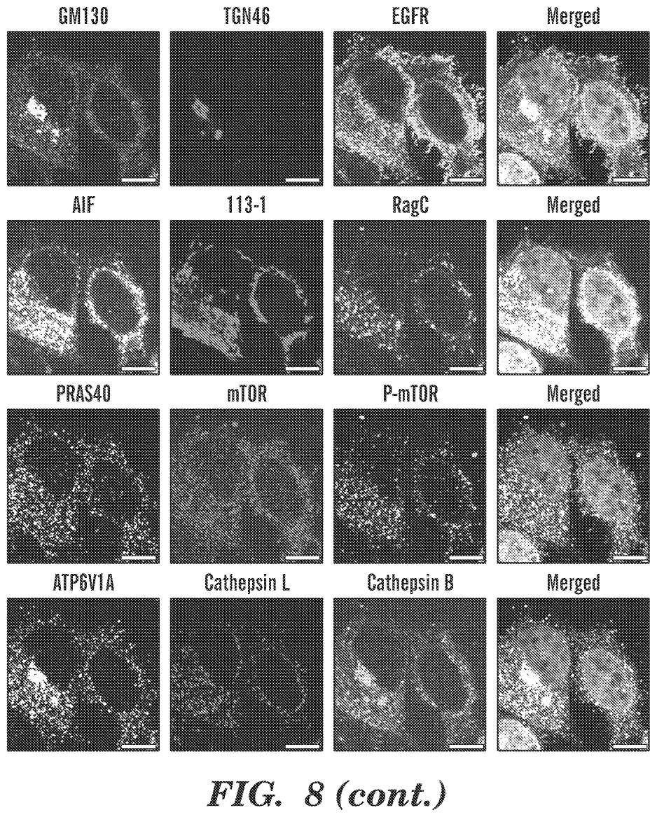

Formaldehyde-fixed Hela cells were incubated overnight at 4° C. with 24 DNA-barcoded antibodies. All DNA amplifiers in 2× SSC+10% dextran sulfate+30% formamide were added for one hour at 37° C. Then, the first three imagers were added in 2× SSC+10% formamide for ten minutes at room temperature. The sample was then imaged on a Nikon Yokogawa CSU-W 1 Spinning Disk Confocal Microscope with a 100× oil objective. After imaging, imagers were stripped by flowing 2×0 SSC+45% formamide over the sample for 15 minutes. The second set of imagers were then added for 5 minutes at room temperature, after which the sample was rinsed with 2× SSC+10% for 5 minutes. The sample was then imaged again, and this cycle was repeated for all imaging rounds. Automatic fluidic exchange was achieved by mounting the sample in a Bioptechs FCS2 chamber hooked up to a Fluigent Aria sequential perfusion system. Results are shown in FIG. 8.

### Example 4: Crosstalk Between SIMPL Channels is Easily Prevented by the Addition of “Cross-Talk Blocker” Strands

Despite the fact that all orthogonal DNA sequences are designed to be “far apart” in sequence space, crosstalk is possible due to the high dynamic range of proteins. If one protein target is highly abundant and another is orders of magnitude less abundant, then even if a DNA imager has high specificity for the corresponding DNA amplifier strand, it may bind to the other strand in an off-target manner. To prevent this, cross-talk blockers, which are identical to imagers but without the fluorophore, can be added at a low concentration to block unused amplifier strands from off-target binding.

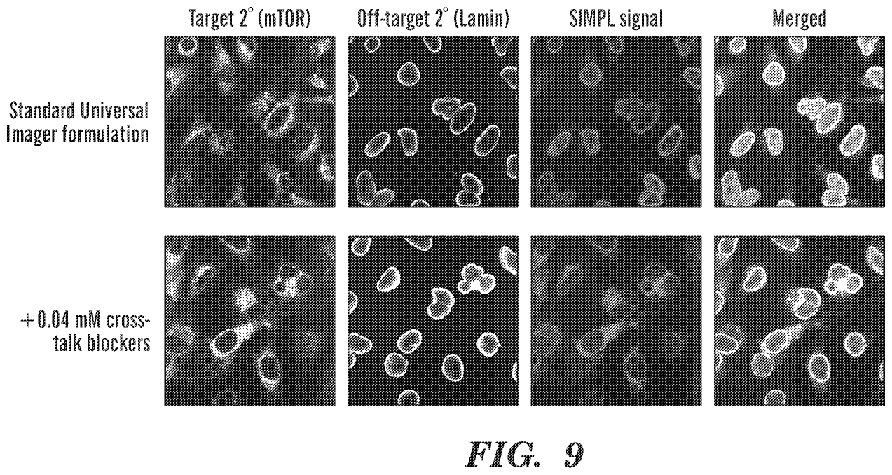

As a proof-of-concept, we stained Hela cells with two antibodies, one targeting a highly abundant target (Lamin A/C) and the other targeting a far less abundant target (mTOR) simultaneously. Formaldehyde-fixed Hela cells were incubated overnight at 4° C. with rabbit anti-mTOR and mouse anti-Lamin A/C antibodies. Both antibodies were DNA-barcoded. The following day, anti-rabbit secondary antibody labeled with Alexa-647 and anti-mouse secondary labeled with Alexa-488 were added for 30 minutes at room temperature. DNA amplifier strands for both barcodes were then added in 2× SSC+10% dextran sulfate+30% formamide were added for one hour at 37° C. Finally, DNA imagers targeting mTOR were added at 0.4 mM in 2× SSC+10% formamide, in the presence or absence of cross-talk blockers at 0.04 mM. Results are shown in FIG. 9. In the absence of cross-talk blockers, imagers targeting mTOR localize to Lamin A/C (SIMPL signal; FIG. 9, top row). In the presence of cross-talk blockers, SIMPL signal is specific to mTOR (SIMPL signal; FIG. 9, bottom row).

### Example 5: SIMPL Multiplexing Enables Complete Signal Removal with Minimal Decay Over 20 Rounds

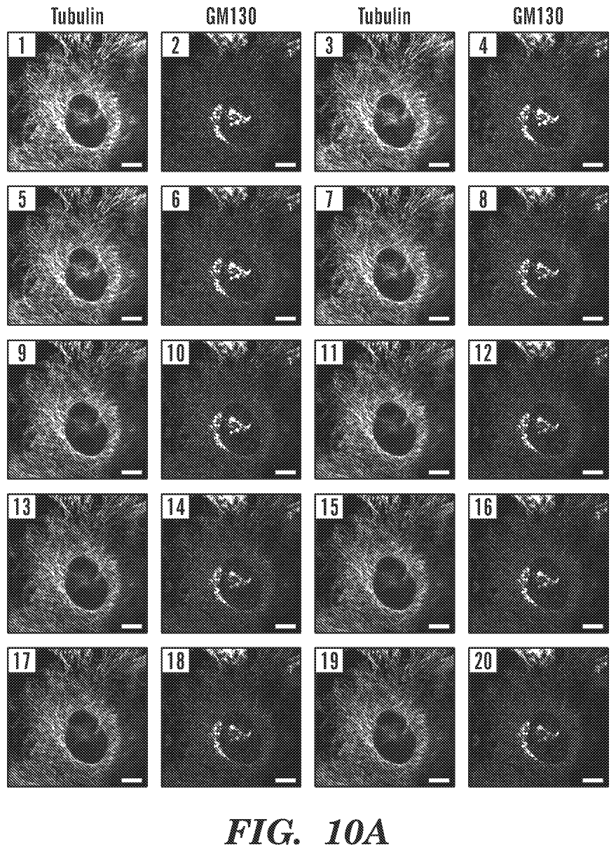

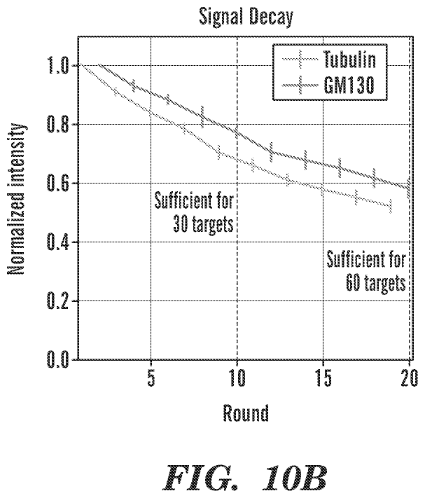

Formaldehyde-fixed Hela cells were incubated overnight at 4° C. with two DNA-barcoded antibodies targeting α-Tubulin and GM130. All DNA amplifiers in 2× SSC+10% dextran sulfate+30% formamide were added for one hour at 37° C. The sample was then imaged on a Nikon Yokogawa CSU-W1 Spinning Disk Confocal Microscope with a 100× oil objective. After imaging, imagers were stripped by flowing 2× SSC+45% formamide over the sample for 15 minutes. The second set of imagers were then added for 5 minutes at room temperature, after which the sample was rinsed with 2× SSC+10% for 5 minutes. The sample was then imaged again, and this cycle was repeated for all imaging rounds. Automatic fluidic exchange was achieved by mounting the sample in a Bioptechs FCS2 chamber hooked up to a Fluigent Aria sequential perfusion system. Results are shown in FIG. 10A.

In FIG. 10B, normalized signal intensity is plotted over 20 rounds. Points represent mean ±standard deviation across all 16 fields of view. After 10 rounds, 70-80% of the signal remained, and after 20 rounds, more than 50% of the signal remained. This corresponds to roughly 2% signal loss per round. 10 rounds of imaging is sufficient to image 30 targets, and 20 rounds of imaging is sufficient to image 60 targets using three fluorescent channels.

### Example 6: SIMPL Multiplexing Enables Many Rounds of RNA Imaging

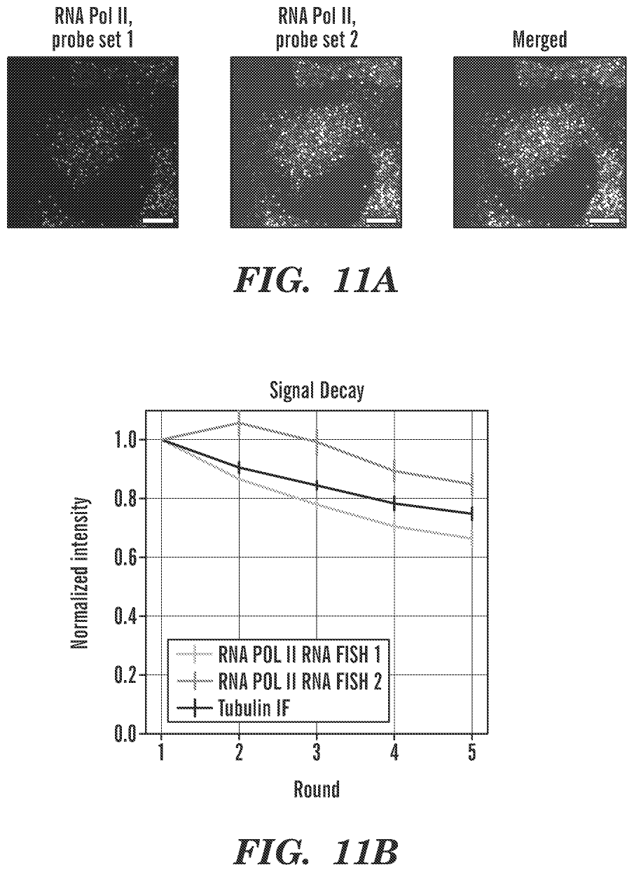

Formaldehyde-fixed Hela cells were incubated overnight at 4° C. with a DNA-barcoded antibody targeting a-Tubulin. Cells were then incubated overnight at 4° C. with two non-overlapping sets of RNA FISH probes targeting the same RNA. Each set has a distinct DNA barcode. All DNA amplifiers in 2× SSC+10% dextran sulfate+30% formamide were added for one hour at 37° C. The sample was then imaged on a Nikon Yokogawa CSU-W1 Spinning Disk Confocal Microscope with a 100× oil objective. After imaging, imagers were stripped by flowing 2× SSC+45% formamide over the sample for 15 minutes. The second set of imagers were then added for 5 minutes at room temperature, after which the sample was rinsed with 2× SSC+10% for 5 minutes. The sample was then imaged again, and this cycle was repeated for all imaging rounds. Automatic fluidic exchange was achieved by mounting the sample in a Bioptechs FCS2 chamber hooked up to a Fluigent Aria sequential perfusion system. FIG. 11A shows maximum intensity projections of the same cell showing the two distinct RNA FISH probe sets. Scale bars are 10 μm. In FIG. 12, normalized signal intensity is plotted over 20 rounds. Points represent mean ±standard deviation across 4 fields of view. RNA signal decayed in an equivalent manner to protein signal, suggesting that SIMPL multiplexing does not strip RNA probes, enabling SIMPL multiplexing to be applied to RNA-FISH.

### References

1. Battich, N., Stoeger, T. and Pelkmans, L., 2013. Image-based transcriptomics in thousands of single human cells at single-molecule resolution. Nature methods, 10(11), pp. 1127-1133.
2. Gerdes, M. J., Sevinsky, C. J., Sood, A., Adak, S., Bello, M. O., Bordwell, A., Can, A., Corwin, A., Dinn, S., Filkins, R. J. and Hollman, D., 2013. Highly multiplexed single-cell analysis of formalin-fixed, paraffin-embedded cancer tissue. Proceedings of the National Academy of Sciences, 110(29), pp. 11982-11987.
3. Jungmann, R., Avendaño, M. S., Woehrstein, J. B., Dai, M., Shih, W. M. and Yin, P., 2014. Multiplexed 3D cellular super-resolution imaging with DNA-PAINT and Exchange-PAINT. Nature methods, 11(3), pp. 313-318.
4. Chen, K. H., Boettiger, A. N., Moffitt, J. R., Wang, S. and Zhuang, X., 2015. Spatially resolved, highly multiplexed RNA profiling in single cells. Science, 348(6233), p. aaa6090.
5. Wang, Y., Woehrstein, J. B., Donoghue, N., Dai, M., Avendaño, M. S., Schackmann, R. C., Zoeller, J. J., Wang, S. S. H., Tillberg, P. W., Park, D. and Lapan, S. W., 2017. Rapid sequential in situ multiplexing with DNA exchange imaging in neuronal cells and tissues. Nano letters, 17(10), pp. 6131-6139.
6. Goltsev, Y., Samusik, N., Kennedy-Darling, J., Bhate, S., Hale, M., Vazquez, G., Black, S. and Nolan, G. P., 2018. Deep profiling of mouse splenic architecture with CODEX multiplexed imaging. Cell, 174(4), pp. 968-981.
7. Gut, G., Herrmann, M. D. and Pelkmans, L., 2018. Multiplexed protein maps link subcellular organization to cellular states. Science, 361(6401), p. eaar7042
8. Lin, J. R., Izar, B., Wang, S., Yapp, C., Mei, S., Shah, P. M., Santagata, S. and Sorger, P. K., 2018. Highly multiplexed immunofluorescence imaging of human tissues and tumors using t-CyCIF and conventional optical microscopes. elife, 7.
9. Eng, C. H. L., Lawson, M., Zhu, Q., Dries, R., Koulena, N., Takei, Y., Yun, J., Cronin, C., Karp, C., Yuan, G. C. and Cai, L., 2019. Transcriptome-scale super-resolved imaging in tissues by RNA seqFISH+. Nature, 568(7751), pp. 235-239.
10. Rouhanifard, S. H., Mellis, I. A., Dunagin, M., Bayatpour, S., Jiang, C. L., Dardani, I., Symmons, O., Emert, B., Torre, E., Cote, A. and Sullivan, A., 2019. ClampFISH detects individual nucleic acid molecules using click chemistry-based amplification. Nature biotechnology, 37(1), pp. 84-89.
11. Kishi, J. Y., Lapan, S. W., Beliveau, B. J., West, E. R., Zhu, A., Sasaki, H. M., Saka, S. K., Wang, Y., Cepko, C. L. and Yin, P., 2019. SABER amplifies FISH: enhanced multiplexed imaging of RNA and DNA in cells and tissues. Nature methods, 16(6), pp. 533-544.
12. Black, S., Phillips, D., Hickey, J. W., Kennedy-Darling, J., Venkataraaman, V. G., Samusik, N., Goltsev, Y., Schürch, C. M. and Nolan, G. P., 2021. CODEX multiplexed tissue imaging with DNA-conjugated antibodies. Nature protocols, 16(8), pp. 3802-3835.
13. Dardani, I., Emert, B.L., Goyal, Y., Jiang, C. L., Kaur, A., Lee, J., Rouhanifard, S. H., Alicea, G. M., Fane, M. E., Xiao, M. and Herlyn, M., 2022. ClampFISH 2.0 enables rapid, scalable amplified RNA detection in situ. Nature methods, 19(11), pp. 1403-1410.
14. He, S., Bhatt, R., Brown, C., Brown, E. A., Buhr, D. L., Chantranuvatana, K., Danaher, P., Dunaway, D., Garrison, R. G., Geiss, G. and Gregory, M. T., 2022. High-plex imaging of RNA and proteins at subcellular resolution in fixed tissue by spatial molecular imaging. Nature Biotechnology, 40(12), pp. 1794-1806.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method comprising:
(a) contacting a sample being tested for the presence of one or more targets with one or more target-specific target-binding molecules, wherein each target-binding molecule is linked to a barcode strand, and wherein target-binding molecules of different specificity are linked to different barcode strands,
(b) optionally removing unbound target-binding molecules,
(c) contacting the sample with amplifier strands comprising a barcode binding domain and a plurality of adapter binding domains, wherein the barcode binding domain comprises a nucleotide sequence that is substantially complementary to a barcode strand,
(d) optionally removing unbound amplifier strands,
(e) contacting the sample with a first set of adaptor strands bound/hybridized with imager strands, wherein the adaptor strands comprise an amplifier binding domain and at least one imager binding domain, wherein the amplifier domain comprises a nucleotide complementary to a nucleotide sequence of an adapter binding domain, and wherein the imager strands comprising a detectable label and a nucleotide sequence that is complementary to a nucleotide sequence of an imager binding domain,
(f) optionally removing unbound adapter strands and imager strands,
(g) imaging the sample to detect location of bound imager strands,
(h) extinguishing a detectable signal from the detectable labels of the bound imager strands, and
(i) optionally, repeating steps (e)-(h) at least once with a second set of adaptor strands bound/hybridized with imager strands and having a unique nucleotide sequence relative to the nucleotide sequences of the adaptor strands in the first set.

2. A method comprising:
(a) contacting a sample being tested for the presence of one or more targets with one or more target-specific target-binding molecules, wherein each target-binding molecule is linked to a barcode strand, and wherein target-binding molecules of different specificity are linked to different barcode strands,
(b) optionally removing unbound target-binding molecules,
(c) contacting the sample with amplifier strands comprising a barcode binding domain and a plurality of adapter binding domains, wherein the barcode binding domain comprises a nucleotide sequence that is substantially complementary to a barcode strand,
(d) optionally removing unbound amplifier strands,
(e) contacting the sample with a first set of adaptor strands comprising an amplifier binding domain and at least one imager binding domain, wherein the amplifier domain comprises a nucleotide complementary to a nucleotide sequence of an adapter binding domain,
(f) optionally removing unbound adapter strands,
(g) contacting the sample with imager strands comprising a detectable label and a nucleotide sequence that is complementary to a nucleotide sequence of an imager binding domain,
(h) optionally removing unbound imager strands,
(i) imaging the sample to detect location of bound imager strands,
(j) extinguishing a detectable signal from the detectable labels of the bound imager strands, and
(k) optionally, repeating steps (e)-(j) at least once with a second set of adaptor strands having a unique nucleotide sequence relative to the nucleotide sequences of the adaptor strands in the first set.

3. The method of any one of the preceding claims, wherein said step of extinguishing the signal from the bound imager strands comprises removing the adaptor strands from the amplifier strands they are hybridized to, or removing the detectable label from the bound imager strands, or modifying the detectable label.

4. The method of any one of the preceding claims, wherein said step of extinguishing the signal from the bound imager strands comprises removing the bound adaptor strands from the amplifier strands they are hybridized to by altering temperature, by altering buffer conditions and/or addition of a complementary nucleic acid strand.

5. The method of any one of the preceding claims, wherein the adaptor strands are removed from the amplifier strand by addition of a denaturant, increasing temperature, addition of a complementary nucleic acid strand, and/or decreasing salt concentration.

6. The method of any one of the preceding claims, wherein the adaptor strands are removed from the amplifier strand by addition of a denaturant selected from the group consisting of formamide, urea, and DMSO.

7. The method of any one of claims 1-5, wherein the adaptor strands are removed from the amplifier strand by addition of complementary nucleic acid strands, wherein the nucleic acid strands comprise a nucleotide sequence substantially complementary to amplifier binding domain of the adaptor strand or the adaptor binding domain of the amplifier strand.

8. The method of any one of claims 1-5, wherein said step of extinguishing the signal from the bound imager strands comprises removing or modifying the detectable label without removing the adaptor strands.

9. The method of any one of claims 1-5 or 8, wherein said step of extinguishing the signal from the bound imager strands comprises photobleaching.

10. The method of any one of claims 1-5 or 8, wherein said step of extinguishing the signal from the bound imager strands comprises cleaving the detectable label from the imager strand.

11. The method of claim 10, wherein said cleaving the detectable label from the imager strand comprises enzymatic cleavage, chemical cleavage or photo cleavage.

12. The method of any one of the preceding claims, wherein at least one adapter strand in the first set of the adaptor strands binds/hybridizes with a first amplifier strand and at least one other adapter strand in the first set of the adaptor strands binds/hybridizes with a second amplifier strand, and wherein the first and second amplifier strand are bound/hybridized to barcode strands linked to target-binding molecules of different specificity (i.e., target-binding molecules bind to different targets).

13. The method of any one of the preceding claims, wherein at least one adapter strand in the second set of the adaptor strands binds/hybridizes with a first amplifier strand and at least one other adapter strand in the second set of the adaptor strands binds/hybridizes with second amplifier strand, and wherein the first and second amplifier strand are bound/hybridized to barcode strands linked to target-binding molecules of different specificity (i.e., target-binding molecules bind to different targets).

14. The method of any one of the preceding claims, wherein at least one imager strand binds to an adaptor strand binds/hybridizes with a first amplifier strand and at least one other adaptor strand binds/hybridizes with a second amplifier strand, and wherein the first and second amplifier strand are bound/hybridized to barcode strands linked to target-binding molecules of different specificity (i.e., target-binding molecules bind to different targets), and wherein the detectable labels of the imager strands are different.

15. The method of any one of the preceding claims, wherein a melting temperature (Tm) of imager strand binding/hybridizing with the adaptor strand is lower than a melting temperature of the amplifier strand binding/hybridizing with the barcode strand and the imager strand binding/hybridizing with the adaptor strand.

16. The method of any one of the preceding claims, wherein a melting temperature (Tm) of imager strand binding/hybridizing with the adaptor strand is at least about 5° C. lower than a melting temperature of the amplifier strand binding/hybridizing with the barcode strand and the imager strand binding/hybridizing with the adaptor strand.

17. The method of any one of the preceding claims, wherein the amplifier binding domain of the adaptor strand is at 5′ of the imager binding domain.

18. The method of any one of claims 1-16, wherein the amplifier binding domain of the adaptor strand is at 3′ of the imager binding domain.

19. The method of any one of the preceding claims, wherein the barcode binding domain of the amplifier strand is 5′ of the plurality of adapter binding domains.

20. The method of any one of claims 1-16, wherein the barcode binding domain of the amplifier strand is 3′ of the plurality of adapter binding domains.

21. A method comprising:
(a) contacting a sample being tested for the presence of one or more targets with one or more target-specific target-binding molecules, wherein each target-binding molecule is linked to a barcode strand, and wherein target-binding molecules of different specificity are linked to different barcode strands,
(b) optionally removing unbound target-binding molecules,
(c) contacting the sample with amplifier strands comprising a barcode binding domain, wherein the barcode binding domain comprises a nucleotide sequence that is substantially complementary to a barcode strand,
(d) optionally removing unbound amplifier strands,
(e) contacting the sample with a first set of imager strands comprising a detectable label and a nucleotide sequence that is substantially complementary to a nucleotide sequence of an imager binding domain,
(f) optionally removing unbound imager strands,
(g) imaging the sample to detect location of bound labeled imager strands,
(h) extinguishing a detectable signal from the bound labeled imager strands, and
(i) optionally, repeating steps (e)-(h), each time with second set of imager strands having a unique nucleotide sequence relative to the imager strands of the first set.

22. The method of claim 21, wherein said step of extinguishing the signal from the bound imager strands comprises removing the imager strands from the amplifier strands they are hybridized to, or removing the detectable label from the bound imager strands, or modifying the detectable label.

23. The method of claim 21 or 22, wherein said step of extinguishing the signal from the bound imager strands comprises removing the bound imager strands from the amplifier strands they are hybridized to by altering temperature, by altering buffer conditions and/or addition of a complementary nucleic acid strand.

24. The method of any one of claims 21-23, wherein the imager strands are removed from the amplifier strand by addition of a denaturant, increasing temperature, addition of a complementary nucleic acid strand, and/or decreasing salt concentration.

25. The method of any one of claims 21-24, wherein the imager strands are removed from the amplifier strand by addition of a denaturant selected from the group consisting of formamide, urea, and DMSO.

26. The method of any one of claims 21-23, wherein the imager strands are removed from the amplifier strand by addition of complementary nucleic acid strands, wherein the nucleic acid strands comprise a nucleotide sequence substantially complementary to amplifier binding domain of the imager strand or the imager binding domain of the amplifier strand

27. The method of any one of claims 21-23, wherein said step of extinguishing the signal from the bound imager strands comprises removing or modifying the detectable label without removing the imager strands.

28. The method of any one of claims 21-23 or 27, wherein said step of extinguishing the signal from the bound imager strands comprises photobleaching.

29. The method of any one of claims 21-23 or 27, wherein said step of extinguishing the signal from the bound imager strands comprises cleaving the detectable label from the imager strand.

30. The method of claim 29, wherein said cleaving the detectable label from the imager strand comprises enzymatic cleavage, chemical cleavage or photo cleavage.

31. The method of any one of claims 21-30, wherein at least one imager strand in the first set of the imager strands binds/hybridizes with a first amplifier strand and at least one other imager strand in the first set of the imager strands binds/hybridizes with second amplifier strand, and wherein the first and second amplifier strand are bound/hybridized to barcode strands linked to target-binding molecules of different specificity (i.e., target-binding molecules bind to different targets), and wherein the detectable labels are different.

32. The method of any one of claims 21-31, wherein at least one imager strand in the second set of the imager strands binds/hybridizes with a first amplifier strand and at least one other imager strand in the second set of the adaptor strands binds/hybridizes with a second amplifier strand, and wherein the first and second amplifier strand are bound/hybridized to barcode strands linked to target-binding molecules of different specificity (i.e., target-binding molecules bind to different targets) and wherein the detectable labels are different.

33. The method of any one of claims 21-32, wherein a melting temperature of imager strand binding/hybridizing with the amplifier strand is lower than the amplifier strand binding/hybridizing with the barcode strand.

34. The method of any one of claims 21-33, wherein a melting temperature of imager strand binding/hybridizing with the amplifier strand is at least about 5° C. lower than the amplifier strand binding/hybridizing with the barcode strand.

35. The method of any one of claims 21-34, wherein the barcode binding domain of the amplifier strand is 5′ of the plurality of imager binding domains. The method of any one of claims 21-34, wherein the barcode binding domain of the amplifier strand is 3′ of the plurality of imager binding domains.

37. The method of any one of the preceding claims, wherein the detectable label is attached at the 5′-end of the imager strand it is attached to.

38. The method of any one of claims 1-36, wherein the detectable label is attached at the 3′- end of the imager strand it is attached to.

39. The method of any one of the preceding claims, wherein the imager strand comprises, at its end the detectable label is attached to, at least two nucleotides that do not hybridized with the strand the imager strand is bound/hybridized to.

40. The method of any one of the preceding claims, wherein the unbound imager strands are partially double-stranded.

41. The method of any one of the preceding claims, wherein the imager strands are molecular beacons or comprise a hairpin secondary structure.

42. The method of any one of the preceding claims, wherein the imager strands are molecular beacons or comprise a hairpin secondary structure that is self-quenching.

43. The method of any one of the preceding claims, wherein the imager strands comprise multiple detectable labels.

44. The method of any one of the preceding clams, wherein the imager strands comprise a first detectable label at their 5′-end and a second detectable label at their 3′-end.

45. The method of any one of claims, wherein the imager strand is from about 10 nucleotides to about 15 nucleotides in length, e.g., about 15 nucleotides in length.

46. The method of any one of the preceding claims, wherein unbound barcode strands are partially double-stranded.

47. The method of any one of the preceding claims, wherein unbound barcode strands comprise a hairpin secondary structure.

48. The method of any one of the preceding claims, wherein the barcode strand is linked to the target-binding molecule via its 5′-end.

49. The method of any one of claims 1-47, wherein the barcode strand is linked to the target-binding molecule via its 3′-end.

50. The method of any one of the preceding claims, wherein the target-binding molecule is an antibody, a nucleic acid, a receptor, a ligand for a receptor, an antigen, or an enzyme.

51. The method of any one of the preceding claims, wherein the sample is contacted with more than one target-binding molecule in step (a).

52. The method of any one of the preceding claims, wherein the target-binding molecule is an antibody, antigen binding portion of an antibody, or a nucleic acid.

53. The method of any one of the preceding claims, wherein the target-binding molecule is an antibody or antigen binding portion of an antibody.

54. The method of any one of the preceding claims, wherein the sample is imaged using confocal or epi-fluorescence microscopy.

55. A composition comprising: (i) a first target-specific target binding molecule linked to a first barcode strand; (ii) a first amplifier strand comprising a barcode binding domain and a plurality of adapter binding domains, wherein the barcode binding domain comprises a nucleotide sequence that is substantially is substantially complementary to the first barcode strand; (iii) at least one first adaptor strand comprising an amplifier binding domain and at least one imager binding domain, wherein the amplifier domain comprises a nucleotide complementary to a nucleotide sequence of the adapter binding domain of the first amplifier strand; and (iv) at least one first imager strand comprising a first detectable label and a nucleotide sequence that is complementary to a nucleotide sequence of the imager binding domain of the first adaptor strand.

56. The composition of claim 55, wherein the composition further comprises (i) a second target-specific target binding molecule linked to a second barcode strand, wherein the second target-binding molecule binds to a target that is different from the first target-binding molecule; (ii) a second amplifier strand comprising a barcode binding domain and a plurality of adapter binding domains, wherein the barcode binding domain comprises a nucleotide sequence that is substantially is substantially complementary to the second barcode strand; (iii) at least one second adaptor strand comprising an amplifier binding domain and at least one imager binding domain, wherein the amplifier domain comprises a nucleotide complementary to a nucleotide sequence of the adapter binding domain of the second amplifier strand; and (iv) at least one second imager strand comprising a second detectable label and a nucleotide sequence that is complementary to a nucleotide sequence of the imager binding domain of the second adaptor strand, and wherein the first and second detectable labels are different.

57. The composition of any one of the preceding claims, wherein a melting temperature of the imager strand binding/hybridizing with the adaptor strand is lower than a melting temperature of the amplifier strand binding/hybridizing with the barcode strand and the imager strand binding/hybridizing with the adaptor strand.

58. The composition of any one of the preceding claims, wherein a melting temperature of imager strand binding/hybridizing with the adaptor strand is at least about 5° C. lower than a melting temperature of the amplifier strand binding/hybridizing with the barcode strand and the imager strand binding/hybridizing with the adaptor strand.

59. The composition of any one of the preceding claims, wherein the amplifier binding domain of the adaptor strand is at 5′ of the imager binding domain. The composition of any one of claims 55-59, wherein the amplifier binding domain of the adaptor strand is at 3′ of the imager binding domain.

61. The composition of any one of the preceding claims, wherein the barcode binding domain of the amplifier strand is 5′ of the plurality of adapter binding domains.

62. The method of any one of claims 55-60, wherein the barcode binding domain of the amplifier strand is 3′ of the plurality of adapter binding domains.

63. The composition of any one of the preceding claims, wherein a melting temperature of the imager strand binding/hybridizing with the adaptor strand is from about 35° C. to about 45° C.

64. The composition of any one of the preceding claims, wherein a melting temperature of the adaptor strand binding/hybridizing with the amplifier strand is at least 50° C. or higher.

65. The composition of any one of the preceding claims, wherein the amplifier strand comprises at least 5 adaptor binding domains.

66. The composition of any one of the preceding claims, wherein the imager strand is hybridized with the adaptor strand.

67. The composition of any one of the preceding claims, wherein the adaptor strand is hybridized with the amplifier strand.

68. A composition comprising: (i) a first target-binding molecule linked to a first barcode strand; (ii) a first amplifier strands comprising a barcode binding domain, wherein the barcode binding domain comprises a nucleotide sequence that is substantially complementary to the first barcode strand; and (iii) a first imager strand comprising a first detectable label and a nucleotide sequence that is substantially complementary to a nucleotide sequence of the first imager binding domain.

69. The composition of claim 68, wherein the composition further comprises: (i) a second target-binding molecule linked to a second barcode strand, wherein the second target-binding molecule binds to a target that is different from the first target-binding molecule; (ii) a second amplifier strands comprising a barcode binding domain, wherein the barcode binding domain comprises a nucleotide sequence that is substantially complementary to the second barcode strand; and (iii) a second imager strand comprising a second detectable label and a nucleotide sequence that is substantially complementary to a nucleotide sequence of the second imager binding domain, and wherein the first and second detectable labels are different.

70. The composition of claim 68 or 69, wherein a melting temperature of imager strand binding/hybridizing with the amplifier strand is lower than the amplifier strand binding/hybridizing with the barcode strand.

71. The composition of any one of claims 68-70, wherein a melting temperature of imager strand binding/hybridizing with the amplifier strand is at least about 5° C. lower than the amplifier strand binding/hybridizing with the barcode strand.

72. The composition of any one of claims 68-71, wherein the barcode binding domain of the amplifier strand is 5′ of the plurality of imager binding domains.

73. The composition of any one of claims 68-72, wherein the barcode binding domain of the amplifier strand is 3′ of the plurality of imager binding domains.

74. The composition of any one of claims 68-73, wherein a melting temperature of the imager strand binding/hybridizing with the amplifier strand is from about 35° C. to about 45° C.

75. The composition of any one of claims 68-74, wherein the amplifier strand comprises at least 5 imager binding domains.

76. The composition of any one of claims 68-75, wherein the imager strand is hybridized with the amplifier strand.

77. The composition of any one of the preceding claims, wherein a melting temperature of the amplifier strand binding/hybridizing with the barcode strand is at least 50° C. or higher.

78. The composition of any one of the preceding claims, wherein the detectable label is attached at the 5′-end of the imager strand it is attached to.

79. The composition of any one of claims 55-78, wherein the detectable label is attached at the 3′-end of the imager strand it is attached to.

80. The composition of any one of the preceding claims, wherein the imager strand comprises, at its end the detectable label is attached to, at least two nucleotides that do not hybridized with the strand the imager strand is bound/hybridized to.

81. The composition of any one of the preceding claims, wherein the unbound imager strands are partially double-stranded.

82. The composition of any one of the preceding claims, wherein the imager strands are molecular beacons or comprise a hairpin secondary structure.

83. The composition of any one of the preceding claims, wherein the imager strands are molecular beacons or comprise a hairpin secondary structure that is self-quenching.

84. The composition of any one of the preceding claims, wherein the imager strands comprise multiple detectable labels.

85. The composition of any one of the preceding claims, wherein the imager strands comprise a first detectable label at their 5′-end and a second detectable label at their 3′-end.

86. The composition of any one of claims, wherein the imager strand is from about 10 nucleotides to about 15 nucleotides in length, e.g., about 15 nucleotides in length.

87. The composition of any one of the preceding claims, wherein unbound barcode strands are partially double-stranded.

88. The composition of any one of the preceding claims, wherein unbound barcode strands comprise a hairpin secondary structure.

89. The composition of any one of the preceding claims, wherein the barcode strand is linked to the target-binding molecule via its 5′-end.

90. The composition of any one of claims 55-89, wherein the barcode strand is linked to the target-binding molecule via its 3′-end.

91. The composition of any one of the preceding claims, wherein the target-binding molecule is an antibody, a nucleic acid, a receptor, a ligand for a receptor, an antigen, or an enzyme.

92. The composition of any one of the preceding claims, wherein the target-binding molecule is an antibody, antigen binding portion of an antibody, or a nucleic acid.

93. The composition of any one of the preceding claims, wherein the target-binding molecule is an antibody or antigen binding portion of an antibody.

94. The composition of any one of the preceding claims, wherein the target-binding molecule is bound to its target.

95. A kit comprising: (i) a first target-specific target binding molecule linked to a first barcode strand; (ii) a first amplifier strand comprising a barcode binding domain and a plurality of adapter binding domains, wherein the barcode binding domain comprises a nucleotide sequence that is substantially is substantially complementary to the first barcode strand; (iii) at least one first adaptor strand comprising an amplifier binding domain and at least one imager binding domain, wherein the amplifier domain comprises a nucleotide complementary to a nucleotide sequence of the adapter binding domain of the first amplifier strand; and (iv) at least one first imager strand comprising a first detectable label and a nucleotide sequence that is complementary to a nucleotide sequence of the imager binding domain of the first adaptor strand.

96. The kit of claim 95, wherein the kit further comprises (i) a second target-specific target binding molecule linked to a second barcode strand, wherein the second target-binding molecule binds to a target that is different from the first target-binding molecule; (ii) a second amplifier strand comprising a barcode binding domain and a plurality of adapter binding domains, wherein the barcode binding domain comprises a nucleotide sequence that is substantially is substantially complementary to the second barcode strand; (iii) at least one second adaptor strand comprising an amplifier binding domain and at least one imager binding domain, wherein the amplifier domain comprises a nucleotide complementary to a nucleotide sequence of the adapter binding domain of the second amplifier strand; and (iv) at least one second imager strand comprising a second detectable label and a nucleotide sequence that is complementary to a nucleotide sequence of the imager binding domain of the second adaptor strand, and wherein the first and second detectable labels are different.

97. The kit of any one of the preceding claims, wherein a melting temperature of the imager strand binding/hybridizing with the adaptor strand is lower than a melting temperature of the amplifier strand binding/hybridizing with the barcode strand and the imager strand binding/hybridizing with the adaptor strand.

98. The kit of any one of the preceding claims, wherein a melting temperature of imager strand binding/hybridizing with the adaptor strand is at least about 5° C. lower than a melting temperature of the amplifier strand binding/hybridizing with the barcode strand and the imager strand binding/hybridizing with the adaptor strand.

99. The kit of any one of the preceding claims, wherein the amplifier binding domain of the adaptor strand is at 5′ of the imager binding domain.

100. The kit of any one of claims 95-98, wherein the amplifier binding domain of the adaptor strand is at 3′ of the imager binding domain.

101. The kit of any one of the preceding claims, wherein the barcode binding domain of the amplifier strand is 5′ of the plurality of adapter binding domains.

102. The method of any one of claims 95-100, wherein the barcode binding domain of the amplifier strand is 3′ of the plurality of adapter binding domains.

103. The kit of any one of the preceding claims, wherein a melting temperature of the imager strand binding/hybridizing with the adaptor strand is from about 35° C. to about 45° C.

104. The kit of any one of the preceding claims, wherein a melting temperature of the adaptor strand binding/hybridizing with the amplifier strand is at least 50° C. or higher.

105. The kit of any one of the preceding claims, wherein the amplifier strand comprises at least 5 adaptor binding domains.

106. The kit of any one of the preceding claims, wherein the imager strand is hybridized with the adaptor strand.

107. The kit of any one of the preceding claims, wherein the adaptor strand is hybridized with the amplifier strand.

108. A kit comprising: (i) a first target-binding molecule linked to a first barcode strand; (ii) a first amplifier strands comprising a barcode binding domain, wherein the barcode binding domain comprises a nucleotide sequence that is substantially complementary to the first barcode strand; and (iii) a first imager strand comprising a first detectable label and a nucleotide sequence that is substantially complementary to a nucleotide sequence of the first imager binding domain.

109. The kit of claim 108, wherein the kit further comprises: (i) a second target-binding molecule linked to a second barcode strand, wherein the second target-binding molecule binds to a target that is different from the first target-binding molecule; (ii) a second amplifier strands comprising a barcode binding domain, wherein the barcode binding domain comprises a nucleotide sequence that is substantially complementary to the second barcode strand; and (iii) a second imager strand comprising a second detectable label and a nucleotide sequence that is substantially complementary to a nucleotide sequence of the second imager binding domain, and wherein the first and second detectable labels are different.

110. The kit of claim 108 or 109, wherein a melting temperature of imager strand binding/hybridizing with the amplifier strand is lower than the amplifier strand binding/hybridizing with the barcode strand.

111. The kit of any one of claims 108-110, wherein a melting temperature of imager strand binding/hybridizing with the amplifier strand is at least about 5° C. lower than the amplifier strand binding/hybridizing with the barcode strand.

112. The kit of any one of claims 108-111, wherein the barcode binding domain of the amplifier strand is 5′ of the plurality of imager binding domains.

113. The kit of any one of claims 108-112, wherein the barcode binding domain of the amplifier strand is 3′ of the plurality of imager binding domains.

114. The kit of any one of claims 108-113, wherein a melting temperature of the imager strand binding/hybridizing with the amplifier strand is from about 35° C. to about 45° C.

115. The kit of any one of claims 108-114, wherein the amplifier strand comprises at least 5 imager binding domains.

116. The kit of any one of claims 108-75, wherein the imager strand is hybridized with the amplifier strand.

117. The kit of any one of the preceding claims, wherein a melting temperature of the amplifier strand binding/hybridizing with the barcode strand is at least 50° C. or higher.

118. The kit of any one of the preceding claims, wherein the detectable label is attached at the 5′-end of the imager strand it is attached to.

119. The kit of any one of claims 95-118, wherein the detectable label is attached at the 3′-end of the imager strand it is attached to.

120. The kit of any one of the preceding claims, wherein the imager strand comprises, at its end the detectable label is attached to, at least two nucleotides that do not hybridized with the strand the imager strand is bound/hybridized to.

121. The kit of any one of the preceding claims, wherein the unbound imager strands are partially double-stranded.

122. The kit of any one of the preceding claims, wherein the imager strands are molecular beacons or comprise a hairpin secondary structure.

123. The kit of any one of the preceding claims, wherein the imager strands are molecular beacons or comprise a hairpin secondary structure that is self-quenching.

124. The kit of any one of the preceding claims, wherein the imager strands comprise multiple detectable labels.

125. The kit of any one of the preceding claims, wherein the imager strands comprise a first detectable label at their 5′-end and a second detectable label at their 3′-end.

126. The kit of any one of claims, wherein the imager strand is from about 10 nucleotides to about 15 nucleotides in length, e.g., about 15 nucleotides in length.

127. The kit of any one of the preceding claims, wherein unbound barcode strands are partially double-stranded.

128. The kit of any one of the preceding claims, wherein unbound barcode strands comprise a hairpin secondary structure.

129. The kit of any one of the preceding claims, wherein the barcode strand is linked to the target-binding molecule via its 5′-end.

130. The kit of any one of claims 95-128, wherein the barcode strand is linked to the target-binding molecule via its 3′-end.

131. The kit of any one of the preceding claims, wherein the target-binding molecule is an antibody, a nucleic acid, a receptor, a ligand for a receptor, an antigen, or an enzyme.

132. The kit of any one of the preceding claims, wherein the target-binding molecule is an antibody, antigen binding portion of an antibody, or a nucleic acid.

133. The kit of any one of the preceding claims, wherein the target-binding molecule is an antibody or antigen binding portion of an antibody.

134. The kit of any one of the preceding claims, wherein the target-binding molecule is bound to its target.

135. The kit of any one of claims 95-134, wherein the kit further comprises a blocker strand.

136. The composition of any one of claims 55-94, wherein the composition further comprises a blocker strands.

137. The method of any one of claims 1-54, wherein the step of contacting the sample with the imager strands is in presence of blocker strands.

138. The method of claim 137, wherein a concentration of the blocker strands is lower than a concentration of the imager strands.
